(19) United States
(12) Patent Application Publication
(10) Pub. No.: US 2024/0156383 A1
(43) Pub. Date: May 16, 2024

(54) MEASUREMENT PROBE, CATHETER SET, AND MEASUREMENT SYSTEM

(71) Applicant: TERUMO KABUSHIKI KAISHA, Tokyo (JP)

(72) Inventor: Satoshi SAWADA, Hadano-shi (JP)

(73) Assignee: TERUMO KABUSHIKI KAISHA, Tokyo (JP)

(21) Appl. No.: 18/417,119

(22) Filed: Jan. 19, 2024

Related U.S. Application Data
(63) Continuation of application No. PCT/JP2022/010536, filed on Mar. 10, 2022.

(30) Foreign Application Priority Data
Jul. 30, 2021 (JP) 2021-126085

(51) Int. Cl. A61B 5/20; A61B 5/00; G01N 33/493

(52) U.S. Cl. CPC A61B 5/201; A61B 5/0062; A61B 5/0071; A61B 5/0075; A61B 5/6852; A61B 5/6874; A61B 5/742; G01N 33/493; A61B 2560/0462; A61B 2562/0233; A61B 2562/043; A61B 2562/185

(57) ABSTRACT

A measurement probe includes: an elongated body that is insertable into a catheter having a flow path; a sensor fixed to the elongated body and capable of detecting a state of a fluid flowing in the flow path; and a sensor holding portion that holds the sensor at a predetermined position when the elongated body is inserted into the catheter. An output of the sensor changes depending on an oxygen partial pressure in the fluid, a carbon dioxide partial pressure in the fluid, a hydrogen ion index of the fluid, an amount of potassium ions in the fluid, an amount of sodium ions in the fluid, an amount of chlorine ions in the fluid, a temperature of the fluid, or a flow rate of the fluid.

### CROSS-REFERENCES TO RELATED APPLICATIONS

This application is a continuation of International Application No. PCT/JP2022/010536 filed on Mar. 10, 2022, which claims priority to Japanese Application No. 2021-126085 filed on Jul. 30, 2021, the entire content of both of which is incorporated herein by reference.

### TECHNOLOGICAL FIELD

The present disclosure generally relates to a measurement probe, a catheter set, and a measurement system.

### BACKGROUND DISCUSSION

Various catheters such as bladder indwelling catheters and intravascular catheters are used in a medical setting. An apparatus has been proposed in which a sensor is inserted into a bladder through a bladder indwelling catheter to measure oxygen in the bladder (Japanese Patent Application Publication No. H9-505505 A).

In the apparatus of Japanese Patent Application Publication No. H9-505505 A, however, the sensor protrudes from a distal end of the catheter. Therefore, there is a possibility that the sensor comes into direct contact with an endothelium or the like of the bladder due to a body motion or the like of a patient, thereby damaging the bladder. Further, it cannot be said that oxygen of urine is correctly measured because a bladder wall is measured.

### SUMMARY

A measurement probe is disclosed, which includes: an elongated body configured to be inserted into a catheter, the catheter having a flow path; a sensor fixed to the elongated body and configured to detect a state of a fluid flowing in the flow path; and a sensor holding portion configured to hold the sensor at a predetermined position when the elongated body is inserted into the catheter.

A catheter set is disclosed, which includes: a catheter, the catheter including an opening provided on a distal end side, and a flow path that is provided between the opening and a rear end side and includes a large diameter portion disposed in the opening, a small diameter portion disposed on the rear end side, and a tapered portion disposed between the large diameter portion and the small diameter portion; and a measurement probe, the measurement probe includes a sensor disposed on the tapered portion.

A measurement system is disclosed, which includes: a measurement probe, the measurement probe includes: an elongated body configured to be inserted into a catheter, the catheter having a flow path; a sensor fixed to the elongated body and configured to detect a state of a fluid flowing in the flow path; and a sensor holding portion configured to hold the sensor at a predetermined position when the elongated body is inserted into the catheter; and a measurement device, the measurement device includes: a data acquisition unit configured to acquire data from the sensor held by the sensor holding portion; a determination unit configured to determine a condition of a patient in which the catheter is indwelled based on the acquired data; and a display unit configured to display the determined condition.

A catheter set comprising: a catheter, the catheter including an opening provided on a distal end side, a flow path provided between the opening and a rear end side, and a probe conduit disposed in parallel with the flow path; a measurement probe, the measurement probe including a light guide that is configured to inserted into the probe conduit; and wherein the catheter further includes a protrusion preventing portion configured to prevent the light guide from protruding from a distal end side of the probe conduit.

A measurement system comprising: a catheter, the catheter includes an opening provided on a distal end side, a flow path provided between the opening and a rear end side, and a probe conduit disposed in parallel with the flow path, a measurement probe; a measurement device; the catheter or the measurement probe includes a sensor configured to detect a state of a fluid flowing in the flow path; the catheter further includes a protrusion preventing portion configured to prevent a distal portion of the measurement probe from protruding from a distal end side of the probe conduit; and the measurement device includes: a data acquisition unit configured to acquire data from the sensor via the measurement probe held in the probe conduit by the protrusion preventing portion; and a display unit configured to display information on a patient in which the catheter is indwelled based on the acquired data.

(1) A measurement probe comprising: an elongated body configured to be inserted into a catheter having a flow path; a sensor fixed to the elongated body and configured to detect a state of a fluid flowing in the flow path; and a sensor holding portion configured to hold the sensor at a predetermined position when the elongated body is inserted into the catheter.

(2) The measurement probe according to (1), wherein an output of the sensor is configured to change depending on an oxygen partial pressure in the fluid, a carbon dioxide partial pressure in the fluid, a hydrogen ion index of the fluid, an amount of potassium ions in the fluid, an amount of sodium ions in the fluid, an amount of chlorine ions in the fluid, a temperature of the fluid, or a flow rate of the fluid.

(3) The measurement probe according to (1) or (2), wherein the sensor holding portion abuts against an end of the flow path.

(4) The measurement probe according to any one of (1) to (3), wherein the sensor includes a light emitter that is configured to emit light when the light emitter comes into contact with a component to be measured, and the elongated body includes a light guide that is configured to guide radiation light emitted from the light emitter.

(5) The measurement probe according to (4), wherein the radiation light is fluorescence.

(6) The measurement probe according to (4) or (5), wherein the light emitter includes a fluorescent dye.

(7) The measurement probe according to any one of (4) to (6), further comprising: a light shield that is configured to prevent light excluding the radiation light from entering the light guide.

(8) The measurement probe according to any one of (4) to (7), wherein a light emission state of the light emitter is configured to change depending on an oxygen partial pressure in the fluid, a carbon dioxide partial pressure in the fluid, a hydrogen ion index of the fluid, an amount of potassium ions in the fluid, an amount of sodium ions in the fluid, an amount of chlorine ions in the fluid, or a temperature of the fluid.

(9) The measurement probe according to any one of (4) to (8), wherein the light emission state of the light emitter is configured to change depending on each of a plurality of measurement items.

(10) The measurement probe according to any one of (1) to (9), comprising a plurality of the sensors fixed to the elongated body.

(11) The measurement probe according to (10), wherein the plurality of the sensors are fixed at different positions along a longitudinal direction of the elongated body.

(12) The measurement probe according to (10), wherein the plurality of the sensors are fixed at a same position along a longitudinal direction of the elongated body.

(13) The measurement probe according to any one of (1) to (12), wherein the plurality of the sensors is configured to detect states related to different measurement items, respectively.

(14) The measurement probe according to any one of (1) to (13), wherein the catheter is a bladder indwelling catheter having a urine passage, and the elongated body is configured to be inserted into the urine passage.

(15) A catheter set comprising: a catheter; and a measurement probe, wherein the catheter includes: an opening provided on a distal end side; a flow path provided between the opening and a rear end side; and a probe conduit disposed in parallel with the flow path, the measurement probe includes a light guide that is configured to be inserted into the probe conduit, and the catheter further includes a protrusion preventing portion that is configured to prevent the light guide from protruding from a distal end side of the probe conduit.

(16) The catheter set according to (15), wherein the probe conduit includes a communication path communicating with the flow path, and the measurement probe includes a sensor fixed to the light guide and configured to detect a state of a fluid flowing in the communication path.

(17) The catheter set according to (15), wherein the catheter is disposed between the flow path and the probe conduit, and includes a sensor configured to detect a state of a fluid flowing in the flow path.

(18) The catheter set according to (15), wherein the probe conduit is open to the distal end side of the catheter, and the measurement probe includes a sensor fixed to the light guide and configured to detect a state of a fluid flowing in the flow path.

(19) The catheter set according to any one of (16) to (18), wherein the sensor includes a light emitter that is configured to emit light when the light emitter comes into contact with a component to be measured, and the light guide is configured to guide radiation light emitted from the light emitter.

(20) The catheter set according to (19), wherein the radiation light is fluorescence.

(21) The catheter set according to (19) or (20), wherein a light emission state of the light emitter is configured to change depending on an oxygen partial pressure in the fluid, a carbon dioxide partial pressure in the fluid, a hydrogen ion index of the fluid, an amount of potassium ions in the fluid, an amount of sodium ions in the fluid, an amount of chlorine ions in the fluid, or a temperature of the fluid.

(22) The catheter set according to any one of (19) to (21), wherein the sensor includes a plurality of the light emitters respectively corresponding to a plurality of measurement items.

(23) The catheter set according to any one of (15) to (22), further comprising: a fixing portion configured to fix the measurement probe in a state of being inserted into the probe conduit.

(24) The catheter set according to claim 23), wherein the fixing portion is configured to seal a space between the measurement probe and the probe conduit.

(25) The catheter set according to any one of (15) to (24), wherein the catheter is a bladder indwelling catheter, and the catheter set further comprising: a urine collection bag connected to the bladder indwelling catheter; and a fastener that is configured to fasten the measurement probe to a surface of the urine collection bag.

(26) A catheter set comprising: a catheter; and a measurement probe, wherein the catheter includes: an opening provided on a distal end side; and a flow path that is provided between the opening and a rear end side and includes a large diameter portion disposed in the opening, a small diameter portion disposed on the rear end side, and a tapered portion disposed between the large diameter portion and the small diameter portion, and the measurement probe includes a sensor disposed on the tapered portion.

(27) The catheter set according to (26), further comprising: a check valve provided in the large diameter portion.

(28) A measurement system comprising: a measurement probe; and a measurement device, wherein the measurement probe includes: an elongated body configured to be inserted into a catheter having a flow path; a sensor fixed to the elongated body and configured to detect a state of a fluid flowing in the flow path; and a sensor holding portion that is configured to hold the sensor at a predetermined position when the elongated body is inserted into the catheter, and the measurement device includes: a data acquisition unit that is configured to acquire data from the sensor held by the sensor holding portion; a determination unit that is configured to determine a condition of a patient in which the catheter is indwelled based on the acquired data; and a display unit that is configured to display the determined condition.

(29) A measurement system comprising: a catheter; a measurement probe; and a measurement device, wherein the catheter includes: an opening provided on a distal end side; a flow path provided between the opening and a rear end side; and a probe conduit disposed in parallel with the flow path, the catheter or the measurement probe includes a sensor configured to detect a state of a fluid flowing in the flow path, the catheter further includes a protrusion preventing portion that is configured to prevent a distal portion of the measurement probe from protruding from a distal end side of the probe conduit, and the measurement device includes: a data acquisition unit that is configured to acquire data from the sensor via the measurement probe held in the probe conduit by the protrusion preventing portion; and a display unit that is configured to display information on a patient in which the catheter is indwelled based on the acquired data.

(30) The measurement system according to (28) or (29), wherein the sensor includes a light emitter that is configured to emit light when the sensor comes into contact with a component to be measured, the measurement probe includes a light guide that is configured to guide radiation light emitted from the light emitter, the measurement device includes: a light source that is configured to irradiate the light emitter with excitation light via the light guide; and a light receiver that is configured to receive the radiation light guided by the light guide; an optical analyzer that is configured to analyze the radiation light, and the data acquisition unit configured to acquire data related to a light emission state of the light emitter from the optical analyzer.

(31) The measurement system according to (30), further comprising: a light shield that is configured to prevent light excluding the radiation light from entering the light guide.

(32) The measurement system according to (30) or (31), wherein a light emission state of the light emitter configured to change depending on an oxygen partial pressure in the fluid, a carbon dioxide partial pressure in the fluid, a hydrogen ion index of the fluid, an amount of potassium ions in the fluid, an amount of sodium ions in the fluid, an amount of chlorine ions in the fluid, or a temperature of the fluid.

(33) The measurement system according to any one of (30) to (32), wherein the radiation light is fluorescence.

(34) The measurement system according to any one of (30) to (33), wherein the sensor includes a plurality of the light emitters respectively corresponding to a plurality of measurement items, and the light guide is configured to guide beams of the radiation light respectively emitted from the plurality of the light emitters in a mixed state.

(35) The measurement system according to any one of (30) to (34), comprising a plurality of the sensors, wherein the light guide is configured to guide beams of the radiation light respectively emitted from the light emitters of the plurality of the sensors in a mixed state.

(36) The measurement system according to (34) or (35), wherein the measurement device includes: a spectroscopic unit that is configured to spectrally disperse the radiation light; and a plurality of the optical analyzers that is configured to analyze beams of light dispersed by the spectroscopic unit.

(37) The measurement system according to (34) or (35), wherein the measurement device includes a filter that is configured to transmit a specific band of the radiation light, and the optical analyzer configured to analyze light transmitted through the filter.

(38) The measurement system according to any one of (28) to (37), wherein the catheter has a non-communicating conduit that is disposed in parallel with the flow path and does not communicate with the flow path, the measurement system further comprising a non-wetted sensor inserted through the non-communicating conduit.

(39) The measurement system according to (38), wherein the non-wetted sensor is a sensor of a laser flowmeter.

(40) The measurement system according to any one of (28) to (39), wherein the display unit is configured to display the information on the patient in time series.

(41) The measurement system according to any one of (28) to (40), wherein the display unit is configured to display an index related to the patient.

In one aspect, it is possible to provide the measurement probe or the like that enables safe measurement.

### BRIEF DESCRIPTION OF THE DRAWINGS

FIG. 1 is an explanatory view illustrating a configuration of a measurement system.

FIG. 2 is an enlarged cross-sectional view of a distal portion of a bladder indwelling catheter into which a measurement probe is inserted.

FIG. 3A is a cross-sectional view taken along line IIIA-IIIA in FIG. 2.

FIG. 3B is a cross-sectional view taken along line IIIB-IIIB in FIG. 2.

FIG. 4A is an explanatory view illustrating a configuration of the measurement probe.

FIG. 4B is a cross-sectional view of a connection hub.

FIG. 5A is a perspective view of a hub rigid member.

FIG. 5B is a perspective view of a hub flexible member.

FIG. 6 is a perspective view of a fastener.

FIG. 7 is an explanatory view illustrating a configuration of a measurement device.

FIG. 8 is a flowchart illustrating flow of processing of a program.

FIG. 9A is a perspective view of a distal portion of a measurement probe of Modification 1-1.

FIG. 9B is an explanatory view illustrating a configuration of a measurement probe of Modification 1-2.

FIG. 9C is a perspective view of a distal portion of a measurement probe of Modification 1-3.

FIG. 10A is a perspective view of a distal portion of a measurement probe of Modification 1-4.

FIG. 10B is a cross-sectional view taken along line XB-XB in FIG. 10A.

FIG. 11A is a perspective view of a distal portion of a measurement probe of Modification 1-5.

FIG. 11B is a front view of a distal portion of a measurement probe of Modification 1-6.

FIG. 12 is an example of a screen of Modification 1-7.

FIG. 13 is an explanatory view illustrating configurations of a measurement probe and a measurement device of Modification 1-8.

FIG. 14A is a cross-sectional view of a connection hub of Embodiment 2.

FIG. 14B is an explanatory view illustrating a method of using the connection hub of Embodiment 2.

FIG. 15 is an explanatory view illustrating a configuration of a measurement device of Embodiment 3.

FIGS. 16A to 16C are time charts illustrating operation of the measurement device of Embodiment 3.

FIGS. 17A to 17C are time charts illustrating operation of a measurement device of Embodiment 4.

FIG. 18 is a perspective view of a distal portion of a measurement probe of Modification 4-1.

FIG. 19 is an enlarged cross-sectional view of a distal portion of a bladder indwelling catheter into which a measurement probe of Modification 4-2 is inserted.

FIG. 20 is an enlarged cross-sectional view of a distal portion of a bladder indwelling catheter into which a measurement probe of Modification 4-3 is inserted.

FIG. 21 is an explanatory view illustrating a configuration of a measurement device of Embodiment 5.

FIGS. 22A to 22C are time charts illustrating operation of the measurement device of Embodiment 5.

FIG. 23 is an explanatory view illustrating a configuration of a measurement device of Embodiment 6.

FIG. 24 is a cross-sectional view of a bladder indwelling catheter of Embodiment 7.

FIG. 25 is a view taken along arrow XXV in FIG. 24.

FIG. 26A is a cross-sectional view of a connection hub of Embodiment 7.

FIG. 26B is an enlarged cross-sectional view of a distal portion of a bladder indwelling catheter into which a measurement probe of Embodiment 7 is inserted.

FIG. 27A is a cross-sectional view taken along line XXVIIA-XXVIIA in FIG. 26B.

FIG. 27B is an enlarged view of a distal portion of a measurement probe of Modification 7-1.

FIG. 28A is an enlarged cross-sectional view of a distal portion of a measurement probe of Modification 7-2.

FIG. 28B is an enlarged cross-sectional view of a distal portion of a measurement probe of Modification 7-3.

FIG. 29 is an enlarged cross-sectional view of a distal portion of a bladder indwelling catheter into which a measurement probe of Modification 7-4 is inserted.

FIG. 30A is an enlarged cross-sectional view of a distal portion of a bladder indwelling catheter into which a measurement probe of Modification 7-5 is inserted.

FIG. 30B is a cross-sectional view taken along line XXXB-XXXB in FIG. 30A.

FIG. 31 is a front view of a distal portion of a bladder indwelling catheter into which a measurement probe of Modification 7-6 is inserted.

FIG. 32A is a cross-sectional view taken along line XXXIIA-XXXIIA in FIG. 31.

FIG. 32B is a cross-sectional view taken along line XXXIIB-XXXIIB in FIG. 31.

FIG. 33 is a perspective view of a light emitter ring.

FIG. 34A is an enlarged cross-sectional view of a distal portion of a bladder indwelling catheter into which a measurement probe of Modification 7-7 is inserted.

FIG. 34B is a cross-sectional view taken along line XXXIVB-XXXIVB in FIG. 34A.

FIG. 35 is an enlarged cross-sectional view of a distal portion of a bladder indwelling catheter into which a measurement probe of Modification 7-8 is inserted.

FIG. 36A is a cross-sectional view taken along line XXXVIA-XXXVIA in FIG. 35.

FIG. 36B is an explanatory view illustrating a method of inserting the measurement probe into a probe conduit of Modification 7-8.

FIG. 37A is an enlarged cross-sectional view of a distal portion of a bladder indwelling catheter into which a measurement probe of Modification 7-9 is inserted.

FIG. 37B is a cross-sectional view taken along line XXXVIIB-XXXVIIB in FIG. 37A.

FIG. 38 is a cross-sectional view of a bladder indwelling catheter of Modification 7-10.

FIG. 39 is a view taken along arrow XXXIX in FIG. 38.

FIG. 40A is a cross-sectional view taken along line XLA-XLA in FIG. 38.

FIG. 40B is a cross-sectional view taken along line XLB-XLB in FIG. 38.

FIG. 41A is a cross-sectional view of a bladder indwelling catheter of Modification 7-11.

FIG. 41B is a cross-sectional view of a bladder indwelling catheter of Modification 7-12.

FIG. 42 is a cross-sectional view of a bladder indwelling catheter of Modification 7-12.

FIG. 43 is a cross-sectional view of a bladder indwelling catheter of Modification 7-13.

FIG. 44 is a view taken along arrow XLIV in FIG. 43.

FIG. 45 is a cross-sectional view taken along line XLV-XLV in FIG. 43.

FIG. 46A is a cross-sectional view of a connection hub of Modification 7-13.

FIG. 46B is a cross-sectional view of a bladder indwelling catheter of Modification 7-14.

FIG. 47 is an explanatory view illustrating a configuration of a measurement system of Embodiment 8.

FIG. 48A is a cross-sectional view of a measurement probe to which an adapter is mounted.

FIG. 48B is a cross-sectional view of the adapter.

FIG. 49 is an explanatory view illustrating a method of using the measurement system of Embodiment 8.

FIG. 50 is an explanatory view illustrating the method of using the measurement system of Embodiment 8.

FIG. 51 is an explanatory view illustrating a configuration of a bladder indwelling catheter of Embodiment 9.

FIG. 52A is an enlarged cross-sectional view of a distal portion of the bladder indwelling catheter of Embodiment 9.

FIG. 52B is an enlarged cross-sectional view of a distal portion of a bladder indwelling catheter of Modification 9-1.

FIG. 53A is an explanatory view illustrating an assembly procedure of the bladder indwelling catheter of Modification 9-1.

FIG. 53B is an explanatory view illustrating the assembly procedure of the bladder indwelling catheter of Modification 9-1.

FIG. 54 is an explanatory view illustrating the assembly procedure of the bladder indwelling catheter of Modification 9-1.

FIG. 55 is a functional block diagram of a measurement system of Embodiment 10.

### DETAILED DESCRIPTION

Set forth below with reference to the accompanying drawings is a detailed description of embodiments of a measurement probe, a catheter set, and a measurement system.

### First Embodiment

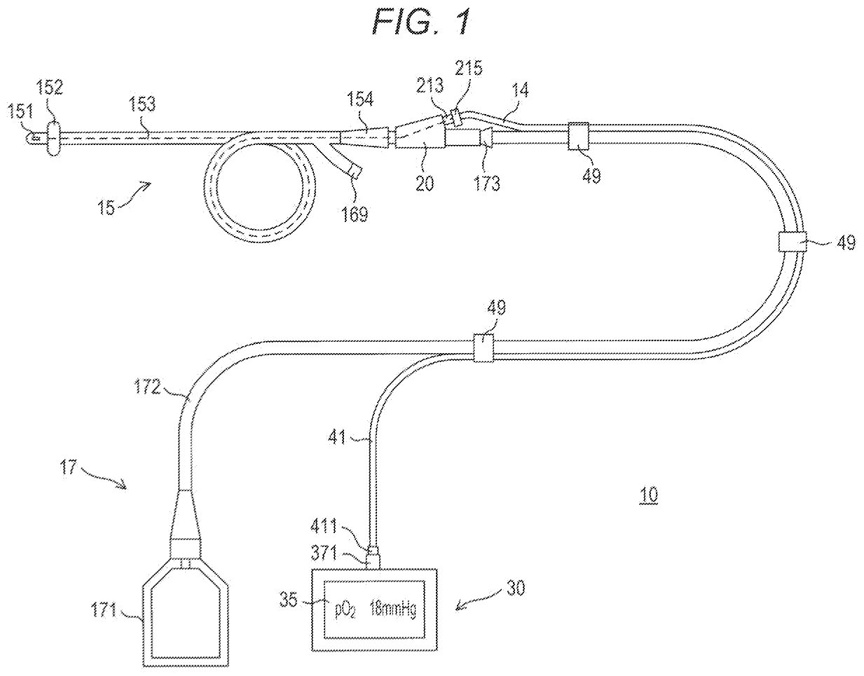

FIG. 1 is an explanatory view illustrating a configuration of a measurement system 10. The measurement system 10 includes a bladder indwelling catheter 15, a measurement probe 14, a connection hub 20, a urine collection bag 17, and a measurement device 30. Note that FIG. 1 is a view schematically illustrating each constituent element of the measurement system 10.

The bladder indwelling catheter 15 includes a shaft 153 having a side hole 151 and a balloon 152 at a distal end, and a urination funnel 154 connected to one end of the shaft 153. The shaft 153 branches into a balloon water injection portion 169 near the urination funnel 154. The urine collection bag 17 includes a bag 171, a urine collection tube 172, and a connection tube 173. The bladder indwelling catheter 15 and the urine collection bag 17 of the present embodiment have been used in related art in a medical setting. Outline of a method of using the bladder indwelling catheter 15 and the urine collection bag 17 in related art will be described.

A user such as a doctor inserts the shaft 153 into a urethra of a patient after connecting the urination funnel 154 and the connection tube 173. In a state where the distal end of the shaft 153 enters the inside of the bladder, the user inflates the balloon 152. The balloon 152 illustrated in FIG. 1 is in an inflated state. When the balloon 152 is inflated, the shaft 153 does not come out of the urethra. The urine of the patient passes through the side hole 151, the shaft 153, and the urine collection tube 172 and is accumulated in the bag 171.

In the present embodiment, as illustrated in FIG. 1, the connection hub 20 is connected between the urination funnel 154 and the connection tube 173. The urine of the patient passes through the side hole 151, the shaft 153, the connection hub 20, and the urine collection tube 172 and is accumulated in the bag 171.

The measurement probe 14 is inserted into the bladder indwelling catheter 15 via the connection hub 20. As indicated by a broken line in FIG. 1, the measurement probe 14 is inserted over substantially the entire length of the shaft 153, and a distal end of the measurement probe 14 is disposed near the side hole 151. The measurement probe 14 includes an optical fiber 41. The optical fiber 41 is provided with an optical fiber connector 411 at a rear end. Details of a configuration of the measurement probe 14 will be described later.

The measurement device 30 can include a display unit 35 and a first connector 371. The optical fiber connector 411 is connected to the first connector 371. The optical fiber 41 can be fixed to the urine collection tube 172 at three points by fasteners 49. In the example illustrated in FIG. 1, an oxygen partial pressure (pO₂) in the urine of the patient is displayed on the display unit 35 in real time.

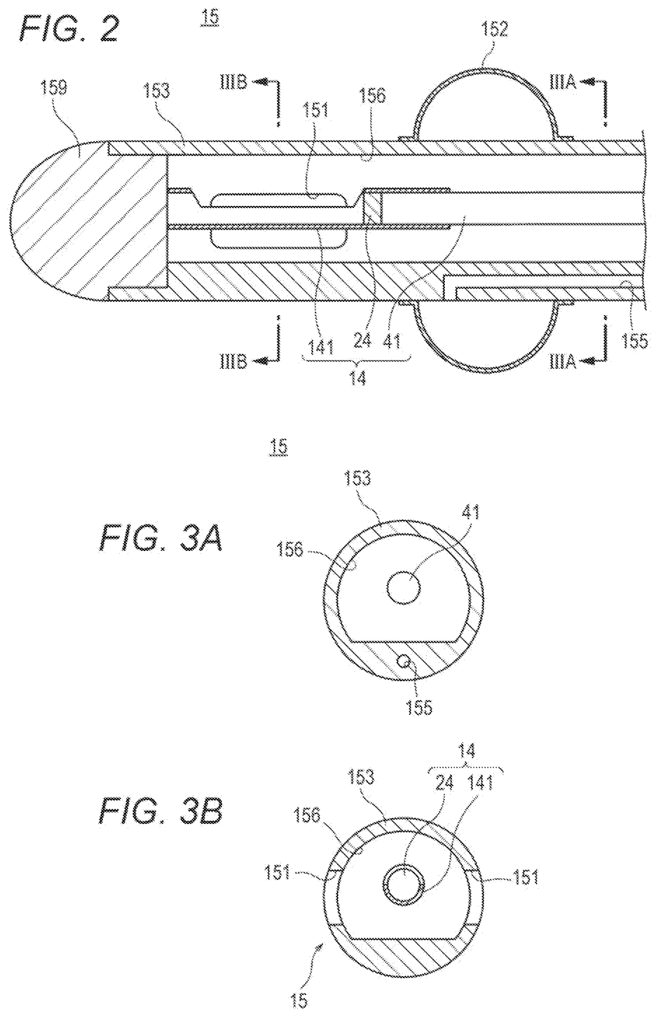

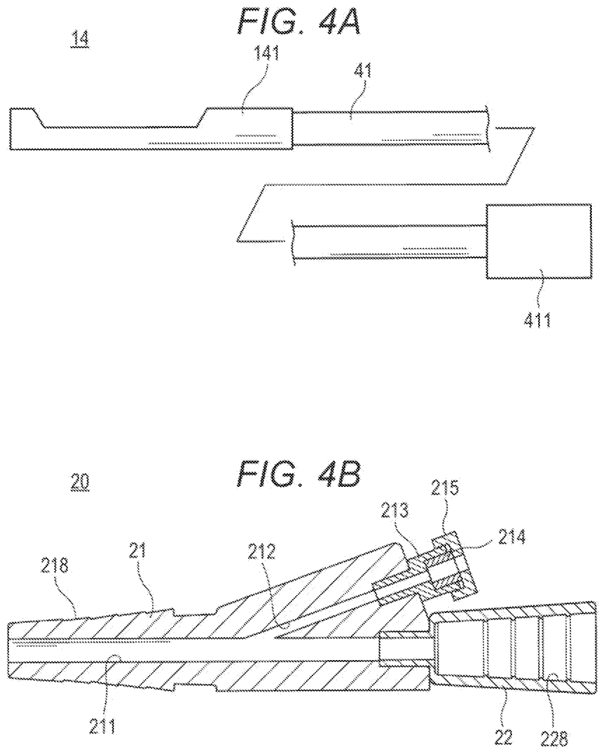

FIG. 2 is an enlarged cross-sectional view of a distal portion of the bladder indwelling catheter 15 into which the measurement probe 14 is inserted. FIG. 3A is a cross-sectional view taken along line IIIA-IIIA in FIG. 2. FIG. 3B is a cross-sectional view taken along line IIIB-IIIB in FIG. 2. FIG. 4A is an explanatory view illustrating the configuration of the measurement probe 14.

As illustrated in FIGS. 2 and 3A, the shaft 153 is a so-called multi-lumen tube having two conduits. The two conduits being a urine passage 156 and a balloon conduit 155. The balloon conduit 155 branches near the urination funnel 154 and penetrates the inside of the balloon water injection portion 169.

The measurement probe 14 is inserted through the urine passage 156. As illustrated in FIG. 2, the balloon conduit 155 is open to the inner side of the balloon 152 and is sealed on a distal end side of such an opening. A distal end of the urine passage 156 is sealed by a distal end member 159. As illustrated in FIG. 3B, a pair of the side holes 151 is provided in the urine passage 156.

As illustrated in FIGS. 2 and 4A, the measurement probe 14 includes a light emitter 24 and a housing 141 in addition to the above-described optical fiber 41 and optical fiber connector 411. The light emitter 24 is disposed at a distal end of the optical fiber 41. Details of the light emitter 24 will be described later. The optical fiber 41 is an example of an elongated body of the present embodiment. The light emitter 24 is an example of a sensor according to the present embodiment.

As illustrated in FIGS. 2 and 3B, the housing 141 has a tubular shape whose side surface is partially cut out (i.e., removed). A distal portion of the optical fiber 41 is inserted into the housing 141. In FIG. 2, a distal end of the housing 141 abuts against the distal end member 159. With the housing 141, the light emitter 24 does not move to the distal end side of the bladder indwelling catheter 15 with respect to the side hole 151. That is, the distal end member 159 implements a function of a protrusion preventing portion that prevents the measurement probe 14 from protruding from the distal end side of the bladder indwelling catheter 15.

That is, the housing 141 implements a function of a sensor holding portion that holds the light emitter 24 on the urination funnel 154 side with respect to the side hole 151. With this configuration, a state in which the light emitter 24 touches fresh urine entering the urine passage 156 from the side hole 151 and flowing toward the urination funnel 154 side can be maintained. The urine passage 156 is an example of a flow path of the present embodiment. The urine is an example of a fluid flowing in the flow path.

The light emitter 24 can be, for example, a translucent resin into which a phosphor is mixed, and can be applied to or molded into a sheet shape and attached to an end surface of the optical fiber 41. The phosphor is an example of a fluorescent dye of the present embodiment. In the present embodiment, a case of using the phosphor that emits fluorescence in response to oxygen in urine will be described as an example. The fluorescence is an example of radiation light to be emitted by the light emitter 24. By analyzing characteristics of the fluorescence emitted from the phosphor, an oxygen partial pressure and an oxygen concentration in the urine can be measured in real time.

Outline of a measurement method using a phosphor will be described. In a case where the phosphor is irradiated with excitation light, the phosphor is in an excited state with high energy. Fluorescence is emitted from the phosphor in the excited state, and the phosphor returns to a ground state. The characteristics of the fluorescence such as intensity, a phase angle, and decay time of the emitted fluorescence change on the basis of a concentration of a quencher with which the phosphor is in contact. Thus, the concentration of the quencher can be measured by analyzing characteristics of the radiation light.

As described above, the phosphor of which the fluorescence characteristics change when being brought into contact with oxygen is used. That is, the quencher in the present embodiment is oxygen. The oxygen partial pressure and the oxygen concentration in the urine can be measured in real time by analyzing the characteristics of the fluorescence in real time.

The radiation light emitted from the phosphor can also include phosphorescence. That is, the measurement may be performed by analyzing characteristics of the phosphorescence. The characteristics of both the fluorescence and the phosphorescence may be analyzed simultaneously.

A phosphor that emits fluorescence in response to carbon dioxide in urine may be used. By analyzing characteristics of fluorescence, a partial pressure of carbon dioxide and a concentration of carbon dioxide in the urine can be measured in real time. A phosphor that emits fluorescence whose characteristics change in accordance with a hydrogen ion index of the urine may be used. By analyzing characteristics of the fluorescence, the hydrogen ion index of the urine, that is, a potential of hydrogen (pH) can be measured in real time.

A phosphor that emits fluorescence in response to ions such as potassium ions, sodium ions or chlorine ions in urine may be used. By analyzing characteristics of the fluorescence, a concentration of an electrolyte in the urine can be measured in real time. In addition, a phosphor that emits fluorescence in response to an arbitrary component in urine may be used.

A phosphor that reacts similarly with respect to a plurality of quenchers may be used. For example, a quencher that is to be brought into contact with the phosphor, that is, a component to be measured can be selected by a diffusion osmosis membrane disposed on a surface of the light emitter 24.

In a phosphor, characteristics of fluorescence also changes depending on a temperature. By analyzing the characteristics of the fluorescence, a temperature of urine can be measured in real time. That is, a plurality of items such as the component in the urine and the temperature of the urine can be simultaneously measured by analyzing the characteristics of radiation light emitted from the light emitter 24.

Note that a side surface of the optical fiber 41 is desirably covered with a sheath. The sheath is desirably a light shield that prevents external light from entering through the side surface of the optical fiber 41. As a result, it is possible to provide the measurement probe 14 in which influence of noise due to the external light can be prevented.

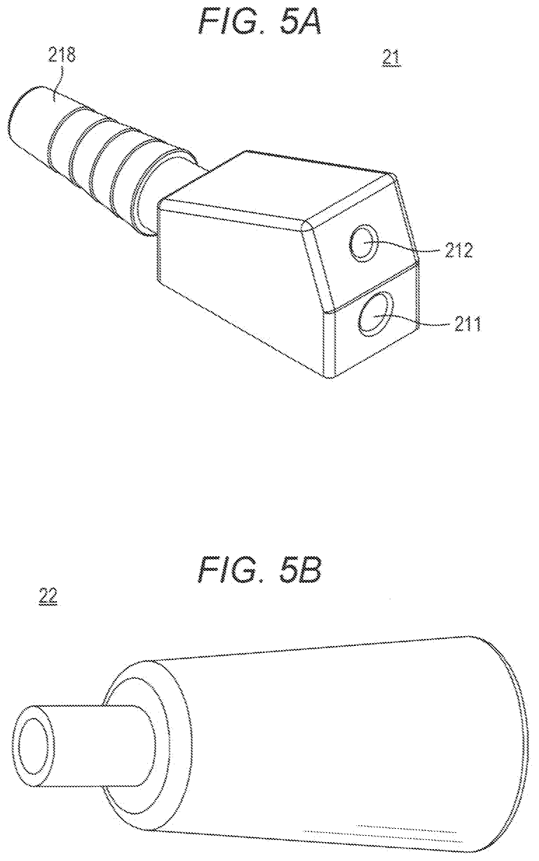

FIG. 4B is a cross-sectional view of the connection hub 20. The connection hub 20 includes a rigid hub member 21, a flexible hub member 22, a holding tube 213, a rubber holding member 214, and a holding lid 215. FIG. 5A is a perspective view of the rigid hub member 21. FIG. 5B is a perspective view of the flexible hub member 22.

The rigid hub member 21 has a catheter connection portion 218 connectable to the urination funnel 154 at one end. The catheter connection portion 218 is provided with protrusions in a stripe shape (i.e., plurality of rings or circular bands) for retraining the urination funnel 154 of the bladder indwelling catheter 15. The catheter connection portion 218 has a size and a shape that can be connected to the urination funnel 154 of the bladder indwelling catheter 15, similarly to the connection tube 173 of the urine collection bag 17 used in related art. A first conduit 211 penetrating the catheter connection portion 218 and a second conduit 212 branching from the first conduit 211 are provided inside the rigid hub member 21.

The flexible hub member 22 is mounted to the other end of the rigid hub member 21. The flexible hub member 22 has a tubular shape and communicates with the first conduit 211. A urine collection bag connection portion 228 connectable to the connection tube 173 is provided on an inner surface of the flexible hub member 22. On a surface of the urine collection bag connection portion 228, protrusions for retaining are provided in a stripe shape (i.e., plurality of rings or circular bands). The flexible hub member 22 is made of rubber or an elastomer and has rubber elasticity. Similarly to the urination funnel 154 of the bladder indwelling catheter 15 in related art, the urine collection bag connection portion 228 has a size and a shape that can be connected to the connection tube 173 of the urine collection bag 17.

The rigid hub member 21 and the flexible hub member 22 are more desirably made of the same material as a material used for a connection portion between the existing bladder indwelling catheter 15 and urine collection bag 17. In these existing instruments, a moldable material such as polyethylene, polycarbonate, polystyrene, nylon, or a nylon resin can be used as a rigid member, and a silicone rubber elastomer, a urethane rubber elastomer, or the like is used as a soft member.

Thus, the connection hub 20 can be mounted between the bladder indwelling catheter 15 and the urine collection bag 17 used in related art. The user can connect the connection hub 20 between the bladder indwelling catheter 15 and the urine collection bag 17 appropriately selected according to a condition of the patient or the like.

The rigid hub member 21 and the flexible hub member 22 are water-tightly fixed by, for example, adhesion or screwing. The rigid hub member 21 and the flexible hub member 22 may be formed by integrally molding different materials.

The holding tube 213 has a tubular shape in which both an outer diameter and an inner diameter on one end side are stepped, and has a male screw portion on the outer periphery of a portion having a large diameter. The rubber holding member 214 has a tubular shape. The holding lid 215 has a cylindrical shape having a through-hole at the center of a bottom portion, and has a female screw portion to be engaged with the male screw portion of the holding tube 213 on an inner surface of a side wall.

In FIG. 4B, the holding lid 215 is loosened. Through-holes respectively provided in the holding lid 215, the rubber holding member 214, and the holding tube 213 communicate with the second conduit 212. After the measurement probe 14 is inserted from the holding lid 215 side, the rubber holding member 214 is compressed to inflate in the radial direction by tightening the holding lid 215. A side surface of the measurement probe 14 is pressed and fixed by the inflating rubber holding member 214. The rubber holding member 214 implements a function of a fixing portion of the present embodiment that is capable of fixing the measurement probe 14 at any position in a state of being inserted into the urine passage 156.

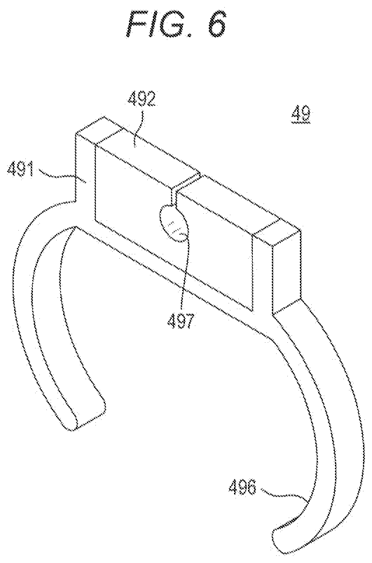

FIG. 6 is a perspective view of the fastener 49. The fastener 49 includes a first part 491 and a second part 492. The first part 491 has a substantially C-shaped urine collection tube holding portion 496. The second part 492 is mounted to the outside of the urine collection tube holding portion 496. The second part 492 includes an optical fiber holding portion 497 disposed at a bottom of a slit.

The urine collection tube holding portion 496 has a size that can be fitted to the outer periphery of the urine collection tube 172. The optical fiber holding portion 497 has a dimension capable of holding the optical fiber 41 pushed in from the slit.

A first material constituting the first part 491 is desirably a material that is relatively hard and easily bendable, such as a hard plastic. A second material constituting the second part 492 is desirably an elastomer such as rubber. The first material may be a material softer than the second material. The first part 491 and the second part 492 may be integrally formed of the same material.

As described with reference to FIG. 1, the optical fiber 41 is fixed to the urine collection tube 172 at a plurality of points by the fasteners 49. The optical fiber 41 may be fixed using, for example, a medical tape, or the like, instead of the fastener 49. The optical fiber 41 may be fixed to any place such as a drip stand or a bed fence.

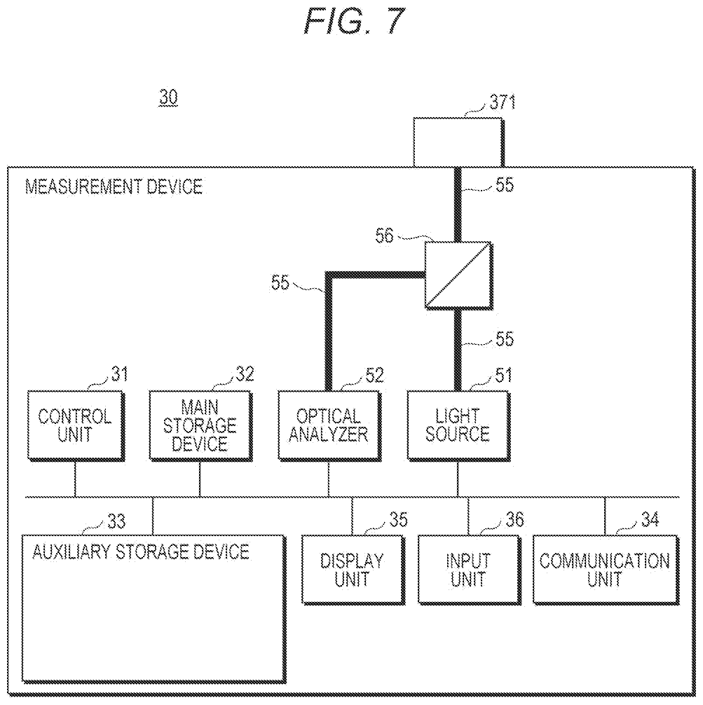

FIG. 7 is an explanatory view illustrating a configuration of the measurement device 30. In addition to the display unit 35 and the first connector 371, the measurement device 30 can include a control unit 31, a main storage device 32, an auxiliary storage device 33, a communication unit 34, an input unit 36, a light source 51, an optical analyzer 52, a light guide path 55, a beam splitter 56, and a bus. The control unit 31 can be an arithmetic control device that executes a program of the present embodiment. For the control unit 31, one or a plurality of central processing units (CPUs), graphics processing units (GPUs), multi-core CPUs, or the like, can be used. The control unit 31 is connected to each hardware unit constituting the measurement device 30 via the bus.

The main storage device 32 is a storage device such as a static random access memory (SRAM), a dynamic random access memory (DRAM), or a flash memory. The main storage device 32 temporarily stores information necessary in the middle of processing to be performed by the control unit 31 and a program being executed by the control unit 31.

The auxiliary storage device 33 is a storage device such as an SRAM, a flash memory, a hard disk, or a magnetic tape. The auxiliary storage device 33 stores a program to be executed by the control unit 31 and various kinds of data necessary for executing the program. The communication unit 34 is an interface that performs communication between the measurement device 30 and a network or other devices.

The display unit 35 can be, for example, a liquid crystal display panel, an organic electro-luminescence (EL) panel, or the like. As illustrated in FIG. 1, the display unit 35 is mounted to a chassis of the measurement device 30. The display unit 35 may be a display device separate from the measurement device 30. For example, a screen of another device such as a biological information monitor may also serve as the display unit 35.

The input unit 36 can be a button, or the like, provided on the chassis of the measurement device 30. The display unit 35 and the input unit 36 may be an integrated panel. The first connector 371 is an optical connector to which the optical fiber 41 is to be connected. The measurement device 30 may include a plurality of the first connectors 371.

The light source 51 can be, for example, a light emitting diode (LED) or a laser diode. The light source 51 irradiates the light emitter 24 with excitation light to excite the phosphor included in the light emitter 24. The excitation light is an example of irradiation light with which the light emitter 24 is irradiated from the light source 51. The light emitted from the light source 51 can include the excitation wavelength of the light emitter 24, however, the wavelength around the wavelength of the fluorescence emitted from the phosphor can be cut off.

The optical analyzer 52 converts the received light into an electrical signal by, for example, a photodiode, and then performs analysis. The light guide path 55 connects between the light source 51 and the beam splitter 56, between the optical analyzer 52 and the beam splitter 56, and between the beam splitter 56 and the first connector 371.

An optical filter that transmits only a wavelength range necessary for exciting the phosphor may be provided in the middle of the light guide path 55 or at an end of the light guide path 55 disposed between the light source 51 and the beam splitter 56. Even in a case where the light source 51 having a wide wavelength range is used, the wavelength range of the excitation light with which the light emitter 24 is irradiated can be precisely selected. Noise due to wavelengths other than the excitation light does not occur, so that it is possible to provide the measurement device 30 with relatively high measurement accuracy.

An optical lens may be disposed in the middle of the light guide path 55 or at an end of the light guide path 55. By effectively using the excitation light and the fluorescence, it is possible to provide the measurement device 30 with relatively high measurement sensitivity.

The measurement device 30 may include a second light source that supplies reference light to the optical analyzer 52 in addition to the light source 51 that emits light for the excitation light. The measurement device 30 may be provided that performs analysis using the reference light. The reference light emitted from the second light source is directly incident on the optical analyzer 52. The second light source and the optical analyzer 52 can be connected by, for example, a dedicated light guide path. A space between the second light source and the optical analyzer 52 may be a cavity configured such that light emitted from the second light source is incident on the optical analyzer 52.

Outline of a method of using the measurement system 10 will be described with reference to FIG. 1. The user connects the bladder indwelling catheter 15, the connection hub 20, and the urine collection bag 17. The user inserts the measurement probe 14 into the bladder indwelling catheter 15 via the connection hub 20. The user can confirm that the housing 141 abuts against the distal end member 159 by the sense of touch or visual observation.

Note that the user may slightly pull back the measurement probe 14 after confirming abutment. When the bladder indwelling catheter 15 is bent, an excessively strong abutment state between the measurement probe 14 and the distal end member 159 provided in the measurement probe 14 can be prevented. Thereafter, the user tightens the holding lid 215 to fix the measurement probe 14. In order to determine an optimum position, a marker may be given in advance to a hand-side portion of the measurement probe, or unevenness that is structurally fitted to the measurement probe may be provided to enable fixing at an appropriate position.

Further, the user may pull back the measurement probe 14 by a predetermined length after confirming that the abutment. The holding tube 213, the rubber holding member 214, and the holding lid 215 of the present embodiment can fix the measurement probe 14 even in a state where the measurement probe 14 is inserted to the middle of the urine passage 156. With this configuration, when the shaft 153 is inserted into the urethra of the patient, damage to the measurement probe 14 due to erroneous gripping of a delicate part, such as the light emitter 24, together with the shaft 153 with forceps or the like can be prevented.

The user inserts the shaft 153 into the urethra of the patient. In a state where the distal end of the shaft 153 enters the inside of the bladder, the user inflates the balloon 152. As described above, the bladder indwelling catheter 15 is indwelled in the patient. The urine of the patient passes through the side hole 151, the urine passage 156, and the urine collection tube 172 and is accumulated in the bag 171.

The user connects the optical fiber connector 411 to the first connector 371. The user fixes the optical fiber 41 to the urine collection tube 172 using the fastener 49.

The description will be continued using FIG. 7. The user operates the measurement device 30 to operate the light source 51. The light emitter 24 is irradiated with the excitation light emitted from the light source 51 through the light guide path 55, the beam splitter 56, and the optical fiber 41. In a case where the light emitter 24 comes into contact with the urine flowing in the urine passage 156, fluorescence corresponding to oxygen in the urine is emitted. The light emitter 24 can function as a sensor capable of detecting an oxygen partial pressure and an oxygen concentration in the urine.

The fluorescence is incident on the beam splitter 56 via the optical fiber 41, the first connector 371, and the light guide path 55. The optical fiber 41 can have a function of propagating light emitted from the light source 51 to the light emitter 24 and light emitted from the light emitter 24. The optical fiber 41 is an example of a light guide of the present embodiment that guides the fluorescence emitted from light emitter 24. The first connector 371 is an example of a light receiver of the present embodiment that receives the fluorescence guided to the optical fiber 41.

The fluorescence enters the beam splitter 56 from the first connector 371 via the light guide path 55. The fluorescence is incident on the light guide path 55 connected to the optical analyzer 52 by the beam splitter 56. The optical analyzer 52 analyzes characteristics of the incident fluorescence and outputs an oxygen partial pressure or an oxygen concentration in the urine to the bus in real time. The control unit 31 displays the oxygen partial pressure in the urine, output from the optical analyzer 52, on the display unit 35.

The user may insert the measurement probe 14 into the bladder indwelling catheter 15 after indwelling the bladder indwelling catheter 15 in the patient. The user can insert the bladder indwelling catheter 15 with relatively high flexibility and a rather easy insertion technique.

For example, the user may insert the measurement probe 14 into the bladder indwelling catheter 15 when observing a condition of the patient and determining that measurement of an oxygen partial pressure in urine is necessary. When the connection hub 20 is connected between the bladder indwelling catheter 15 and the urine collection bag 17, the user can rather quickly use the measurement probe 14 according to the condition of the patient.

When the bladder indwelling catheter 15 is inserted into the patient in a state where the measurement probe 14 is not inserted, a dummy probe having substantially the same outer shape as the measurement probe 14 is desirably inserted into the holding tube 213 and fixed by the rubber holding member 214 and the holding lid 215. A length of the dummy probe may be such a length that the dummy probe reaches the middle of the second conduit 212.

Instead of the holding tube 213, the rubber holding member 214, and the holding lid 215, a valve that maintains watertightness and airtightness in a case where the measurement probe 14 is not inserted may be mounted to the rigid hub member 21. In this manner, even in the state where the measurement probe 14 is not inserted, it is possible to prevent urine of the patient from leaking from the second conduit 212 and prevent bacteria and the like from entering the bladder of the patient via the second conduit 212 to cause infection by maintaining the sealed state of the second conduit 212.

The bladder indwelling catheter 15 and the connection hub 20 may be integrated. Specifically, the urination funnel 154 may have a function of the connection hub 20. The bladder indwelling catheter 15, the connection hub 20, and the urine collection bag 17 may be a so-called closed type supplied to the user in a state of being connected in advance. Furthermore, the measurement probe 14 may be supplied to the user in a state of being inserted into the bladder indwelling catheter 15.

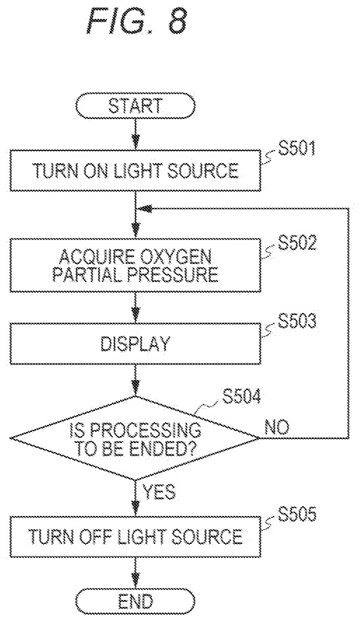

FIG. 8 is a flowchart illustrating flow of processing of the program. The control unit 31 starts the program of FIG. 8 in a case where the user gives an instruction to operate the light source 51.

The control unit 31 turns ON the light source 51 (S501). The light emitter 24 is irradiated with excitation light via the beam splitter 56 and the optical fiber 41. Fluorescence emitted from the phosphor of the light emitter 24 is incident on the optical analyzer 52 via the optical fiber 41 and the beam splitter 56.

The optical analyzer 52 outputs urinary oxygen partial pressure data on the basis of obtained fluorescence characteristics. The control unit 31 acquires the urinary oxygen partial pressure data from the optical analyzer 52 (S502). By S502, the control unit 31 implements a function of a data acquisition unit that acquires data from the sensor held in the sensor holding portion.

As exemplified in FIG. 1, the control unit 31 displays the urinary oxygen partial pressure on the display unit 35 (S503). The control unit 31 determines whether or not to end the processing (S504). For example, the control unit 31 determines to end the processing in a case where operation to turn OFF the light source 51 is received or in a case where the optical fiber 41 is detached from the first connector 371.

In a case where it is determined not to end the processing (NO in S504), the processing of the control unit 31 returns to S502. In a case where it is determined to end the processing (Yes in S504), the control unit 31 turns OFF the light source 51 (S505). The control unit 31 ends the processing.

According to the present embodiment, it is possible to provide the measurement system 10 capable of performing measurement without causing the distal end of the measurement probe 14 to protrude from the bladder indwelling catheter 15. For example, even in a case where a posture of the patient changes, it is possible to prevent the distal end of the measurement probe 14 from damaging a bladder wall.

According to the present embodiment, the light emitter 24 is disposed near the side hole 151 and downstream of the side hole 151. Thus, it is possible to provide the measurement system 10 capable of measuring an oxygen partial pressure and the like in fresh urine just discharged from the bladder to the catheter in real time. Note that the oxygen partial pressure and the like in the urine also change while the urine passes through the inside of the bladder indwelling catheter 15. It is possible to provide the measurement system 10 capable of accurately measuring the change in the oxygen partial pressure by measuring the oxygen partial pressure and the like in the fresh urine in real time.

By measuring the oxygen partial pressure in the urine in real time, signs leading to acute kidney injury can be found relatively early. As compared with a method in related art using a urine amount and a serum creatine level, or a biomarker, a risk of injury due to hypoxia of the kidney using the measurement probe 14 as disclosed can be found at an earlier stage and more appropriate treatment can be performed.

According to the present embodiment, it is possible to provide the measurement probe 14 in which the light emitter 24 is protected by the housing 141. The presence of the housing 141 can help prevent damage to the measurement probe 14 caused by detachment of the light emitter 24 due to contact with an inner surface of the bladder indwelling catheter 15 and the distal end member 159. When the user inserts the measurement probe 14 into the bladder indwelling catheter 15, it is also possible to help prevent damage to the measurement probe 14 caused by the light emitter 24 touching the user's finger, a medical instrument, or the connection hub 20.

According to the present embodiment, it is possible to provide the measurement probe 14 and the connection hub 20 which enable measurement of the oxygen partial pressure and the like in the urine in real time in combination with the existing bladder indwelling catheter 15 and urine collection bag 17. The user can use the bladder indwelling catheter 15 selected on the basis of the condition of the patient, past experience, expertise, and the like in combination with the measurement probe 14 and the connection hub 20 of the present embodiment.

By mounting only the connection hub 20 between the bladder indwelling catheter 15 and the urine collection bag 17, it is possible to provide the measurement system 10 in which the measurement probe 14 can be quickly inserted and used when necessary. Medical cost can be reduced by using the measurement probe 14, which is more expensive than the connection hub 20, only for a necessary patient.

The control unit 31 may give a notification to the user, for example, in a case where the oxygen partial pressure in the urine becomes equal to or lower than a threshold value. For example, the control unit 31 may give the notification to the user by display on the display unit 35, or sound output from the measurement device 30. The control unit 31 may transmit the notification to a nurse station, or the like, via a network such as a hospital information system (HIS) or an electronic medical record (EMR).

For example, the control unit 31 may calculate an index representing a state of a urinary organ such as a kidney on the basis of the oxygen partial pressure in the urine and the temperature and display the index on the display unit 35. The index representing the state of the urinary organ may be calculated by combining information acquired from another device such as a biological information monitor with the oxygen partial pressure in the urine and the temperature. The index is not limited to the index representing the state of the urinary organ and may be an index representing a general condition of the patient.

The optical analyzer 52 is not limited to one having a function of outputting the urinary oxygen partial pressure data on the basis of the obtained fluorescence characteristics. For example, data indicating the fluorescence characteristics such as intensity, a phase angle, and decay time of the received fluorescence, that is, data before calculation of the urinary oxygen partial pressure data may be output to the bus. In such a case, the control unit 31 calculates a urinary oxygen partial pressure, a urinary oxygen concentration, or the like.

An optical analysis block including the light source 51, the optical analyzer 52, the light guide path 55, the beam splitter 56, and the first connector 371 may be separate from the measurement device 30.

In a case where the optical analysis block is separate, the measurement device 30 of the present embodiment may be configured by combining the optical analysis block and a general-purpose information processing device such as a personal computer, a tablet, or a smartphone. In such a case, the optical analysis block can be connected to the information processing device, for example, in a wired or wireless manner.

Note that the display of the display unit 35 illustrated in FIG. 1 is an example. For example, in a case where the light emitter 24 has a phosphor that reacts with potassium ions, the measurement device 30 displays a potassium ion concentration or an amount of potassium ions in the urine on the display unit 35 in real time. Further, the phosphor that reacts with potassium ions and the phosphor that reacts with oxygen may be simultaneously mixed into a structure such as one sheet or a member to be applied to the distal end of the optical fiber 41. Then, it is also possible to obtain a system in which two components are simultaneously measured by one fiber and displayed.

The measurement probe 14 and the connection hub 20 are desirably single use products supplied to the user in a sterilized state, which can help reduce a risk of occurrence of urinary tract infection. A kit in which the measurement probe 14 and the connection hub 20 are combined one by one may be supplied to the user. A catheter set in which the measurement probe 14 is combined with the connection hub 20 and the bladder indwelling catheter 15 may be supplied to the user.

The bladder indwelling catheter 15 is an example of a catheter of the present embodiment. The measurement probe 14 and the connection hub 20 may be mounted to any tube to be used for continuously discharging a body fluid or the like from the patient, such as a chest drainage tube, an abdominal drainage tube, or a brain drainage tube, instead of the bladder indwelling catheter 15. The measurement probe 14 and the connection hub 20 may be mounted to any medical tube used for feeding a liquid into the body of the patient, such as an infusion tube or a feeding tube. These medical tubes are also examples of the catheter of the present embodiment.

### Modification 1-1

The present modification relates to the measurement probe 14 using a coil 142 instead of the housing 141. Description of portions common to Embodiment 1 will be omitted.

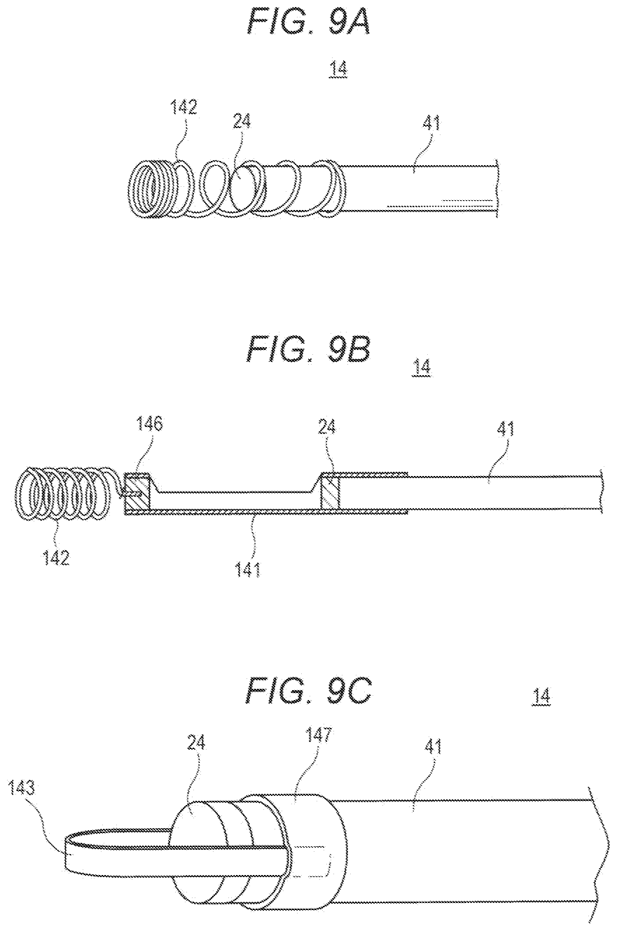

FIG. 9A is a perspective view of a distal portion of the measurement probe 14 of Modification 1-1. The coil 142 is mounted to a distal end of the optical fiber 41. A pitch of the coil 142 can be, for example, several millimeters or less, and a user can be prevented from unintentionally touching the light emitter 24.

According to the present modification, it is possible to provide the measurement probe 14 that is less likely to be damaged even in a case where the measurement probe 14 is pressed against a distal end side of the bladder indwelling catheter 15 due to a change in a posture of a patient or the like. That is, the coil 142 implements a function of a sensor holding portion that holds the light emitter 24 on the urination funnel 154 side with respect to the side hole 151.

### Modification 1-2

The present modification relates to the measurement probe 14 in which the coil 142 is provided at a distal end of the housing 141. Description of portions common to Embodiment 1 will be omitted.

FIG. 9B is an explanatory view illustrating a configuration of the measurement probe 14 of Modification 1-2. The distal end of the housing 141 is sealed by a coil holder 146. The coil 142 is fixed to the coil holder 146. In FIG. 9B, a distal end side of the coil 142 is illustrated in a perspective view, and the other portions are illustrated in a cross-sectional view.

According to the present modification, the measurement probe 14 in which the coil 142 is disposed at the distal end can be relatively easily manufactured. In the present modification, the housing 141 and the coil 142 implement a function of a sensor holding portion that holds the light emitter 24 on the urination funnel 154 side with respect to the side hole 151.

### Modification 1-3

The present modification relates to the measurement probe 14 using a sheet-shaped cover plate 143 instead of the housing 141. Description of portions common to Embodiment 1 will be omitted.

FIG. 9C is a perspective view of a distal portion of the measurement probe 14 of Modification 1-3. The sheet-shaped cover plate 143 bent in a substantially U shape is mounted to a distal end of the optical fiber 41 by a holding tube 147.

The cover plate 143 can be formed by bending a resin sheet, for example. The cover plate 143 may be manufactured by cutting a rod extrusion-molded to have a U-shaped cross section. The holding tube 147 can be, for example, a heat-shrinkable tube. The cover plate 143 may be fixed to the optical fiber 41 by winding a tape with an adhesive, instead of the holding tube 147. The holding tube 147 may cover a joint surface between the light emitter 24 and the optical fiber 41 and a side surface of the light emitter 24.

According to the present modification, it is possible to provide the measurement probe 14 that protects the light emitter 24 with a relatively simple configuration. The cover plate 143 implements a function of a sensor holding portion that holds the light emitter 24 on the urination funnel 154 side with respect to the side hole 151.

### Modification 1-4

The present modification relates to the measurement probe 14 using a wire 144 instead of the housing 141. Description of portions common to Embodiment 1 will be omitted.

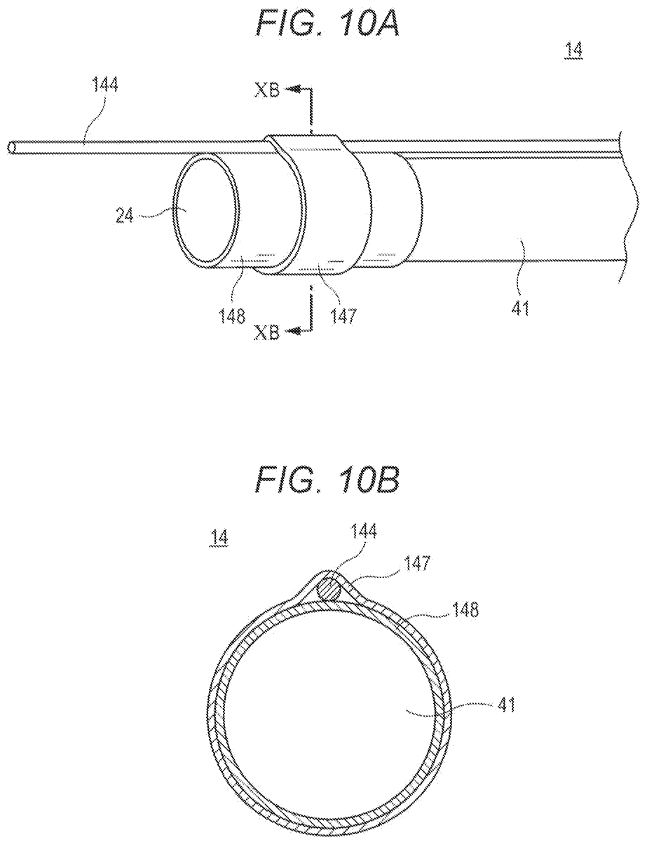

FIG. 10A is a perspective view of a distal portion of the measurement probe 14 of Modification 1-4. FIG. 10B is a cross-sectional view taken along line XB-XB in FIG. 10A. A two-layer tube of a protective tube 148 and the holding tube 147 is mounted to a distal portion of the optical fiber 41. The wire 144 is sandwiched between the protective tube 148 and the holding tube 147.

The protective tube 148 covers a side surface of the light emitter 24 together with the distal portion of the optical fiber 41. The holding tube 147 is shorter than the protective tube 148. The protective tube 148 and the holding tube 147 can be disposed at a plurality of places over the entire length of the optical fiber 41 so as to fix the wire 144 along the optical fiber 41.

According to the present modification, since the protective tube 148 is disposed between the optical fiber 41 and the wire 144, it is possible to prevent the optical fiber 41 from being damaged by the wire 144. Since the wire 144 is disposed along the optical fiber 41, buckling or the like is unlikely to occur when the optical fiber 41 is inserted into the bladder indwelling catheter 15, and it is possible to provide the measurement probe 14 which is relatively easy for a user to handle. The wire 144 implements a function of a sensor holding portion that holds the light emitter 24 on the urination funnel 154 side with respect to the side hole 151.

### Modification 1-5

The present modification relates to the measurement probe 14 to which a temperature sensor 45 is mounted. Description of portions common to Modification 1-4 will be omitted.

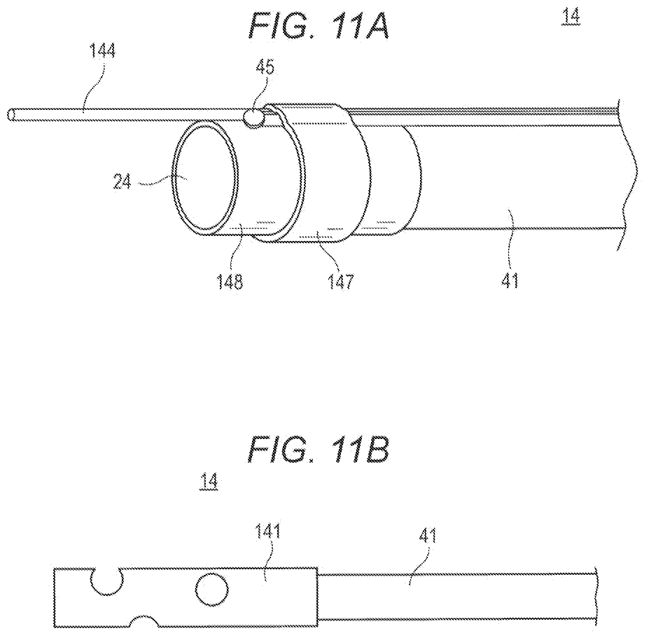

FIG. 11A is a perspective view of a distal portion of the measurement probe 14 of Modification 1-5. The temperature sensor 45 is fixed near the light emitter 24. The temperature sensor 45 can be, for example, a resistance temperature detector, a thermistor, or a thermocouple. A cable of the temperature sensor 45 is fixed to the optical fiber 41 together with the wire 144.

The temperature sensor 45 can be connected to a temperature measuring device. The temperature measuring device may be built in the measurement device 30.

According to the present modification, it is possible to provide the measurement system 10 capable of measuring a temperature by the temperature sensor 45 independent of the light emitter 24. For example, it is possible to provide the measurement device 30 that performs highly accurate measurement by correcting a measurement result measured by the light emitter 24 using the temperature measured by the temperature sensor 45.

### Modification 1-6

The present modification relates to the measurement probe 14 provided with a plurality of openings in the housing 141. Description of portions common to Embodiment 1 will be omitted.

FIG. 11B is a front view of a distal portion of the measurement probe 14 of Modification 1-6. The housing 141 of the present modification is a tube having a plurality of holes. FIG. 11B illustrates a case where the hole is circular, but the hole may have any shape such as an elliptical shape or a rectangular shape.

According to the present modification, it is possible to provide the measurement system 10 in which a measurement result is hardly affected even in a case where the measurement probe 14 has rotated in the bladder indwelling catheter 15.

### Modification 1-7

The present modification relates to the measurement device 30 that displays time-series data on the display unit 35. Description of portions common to Embodiment 1 will be omitted.

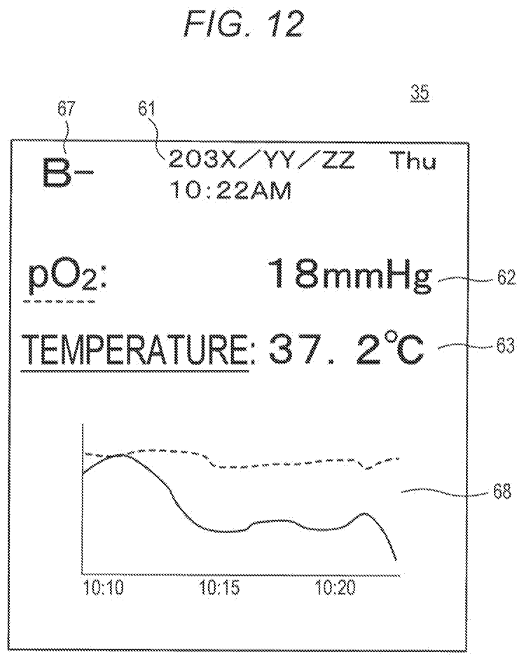

FIG. 12 is an example of a screen of Modification 1-7. In the present modification, the measurement device 30 measures an oxygen partial pressure and a temperature in real time. The measurement device 30 of the present modification includes the display unit 35 that is relatively large.

An index field 67, a date and time field 61, an oxygen partial pressure field 62, a temperature field 63, and a graph field 68 are displayed on the screen. In the index field 67, an index representing a state of a kidney is displayed. Combination of an alphabet and a symbol “+” or “−” allows a user to rather easily grasp the state of the kidney of a patient.

The date, day of the week, and time can be displayed in the date and time field 61. An oxygen partial pressure in urine is displayed in the oxygen partial pressure field 62. A temperature is displayed in the temperature field 63. In the graph field 68, time-series data of each of the oxygen partial pressure in the urine and the temperature is displayed by a line graph. In the graph field 68, a broken line indicates the time-series data of the oxygen partial pressure in the urine, and a solid line indicates the time-series data of the temperature. A broken line displayed under characters of “pO₂” and a solid line displayed under characters of “temperature” in the oxygen partial pressure field 62 function as so-called legend fields. This allows the user to rather easily grasp what the graph means.

The line graph illustrated in the graph field 68 is an example of a graph format. Any form of graph that is relatively easy for the user to use in a clinical setting can be used in the graph field 68. For example, in a case where importance is placed on a value per unit time, a bar graph is used for display in the graph field 68. The user may be able to appropriately specify the format of the graph.

The time-series data may be indicated in a tabular format instead of the graph format. The control unit 31 may appropriately receive setting change of items and a layout to be displayed on the display unit 35 by the user. The user can use the measurement system 10 with user-friendly settings depending on the situation.

### Modification 1-8

The present modification relates to the measurement probe 14 in which the optical fiber 41 is divided into a fiber for irradiation light and a fiber for light reception. Description of portions common to Embodiment 1 will be omitted.

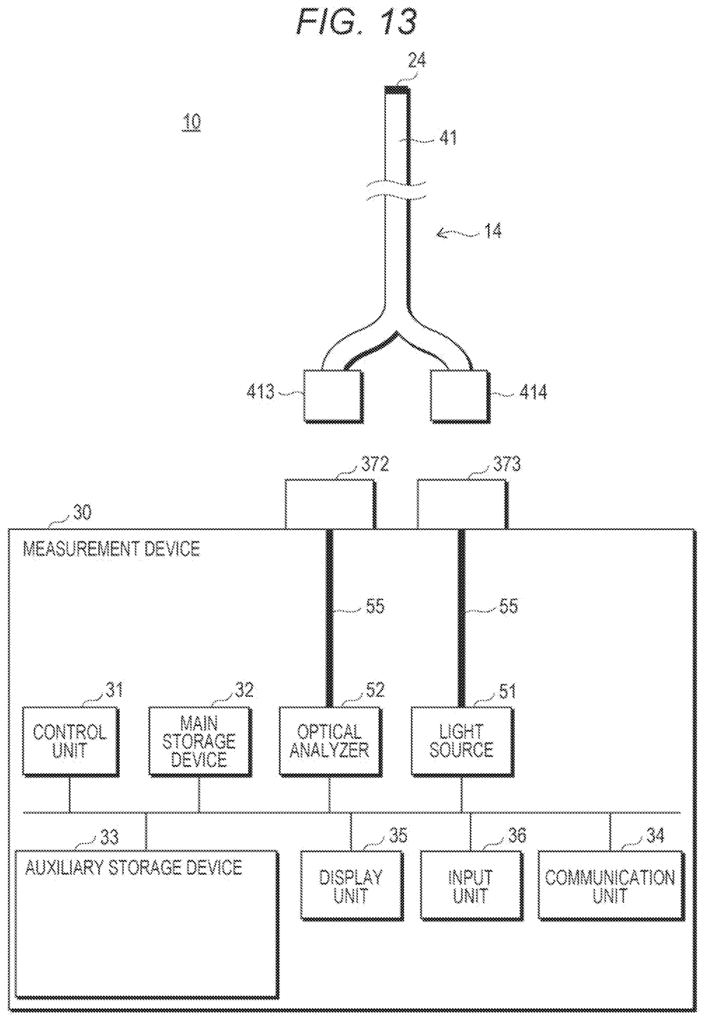

FIG. 13 is an explanatory view illustrating configurations of the measurement probe 14 and the measurement device 30 of Modification 1-8. The optical fiber 41 of the measurement probe 14 is divided into two bundles at an end, and a fluorescence connector 413 is connected to one bundle and an irradiation light connector 414 is connected to the other bundle.

The measurement device 30 includes a second connector 372 and a third connector 373 instead of the first connector 371. The second connector 372 is connected to the optical analyzer 52 via the light guide path 55. The third connector 373 is connected to the light source 51 via the light guide path 55.

The light emitter 24 is irradiated with excitation light emitted from the light source 51 through the light guide path 55, the third connector 373, and the irradiation light connector 414. Fluorescence emitted from the light emitter 24 enters the optical analyzer 52 via the optical fiber 41, the fluorescence connector 413, the second connector 372, and the light guide path 55.

It is desirable to use a fiber, which has specifications suitable for propagation of excitation light, for a fiber connected to the irradiation light connector 414, and a fiber, which has specifications suitable for propagation of fluorescence, can be used for a fiber connected to the fluorescence connector 413.

An optical connector that connects one bundle of fibers and two bundles of fibers may be used for a branch portion at which one bundle of the optical fibers 41 branches into two bundles.

### Embodiment 2

The present embodiment relates to the measurement system 10 suitable in a case where measurement at a junction of the first conduit 211 and the second conduit 212. Description of portions common to Embodiment 1 will be omitted.

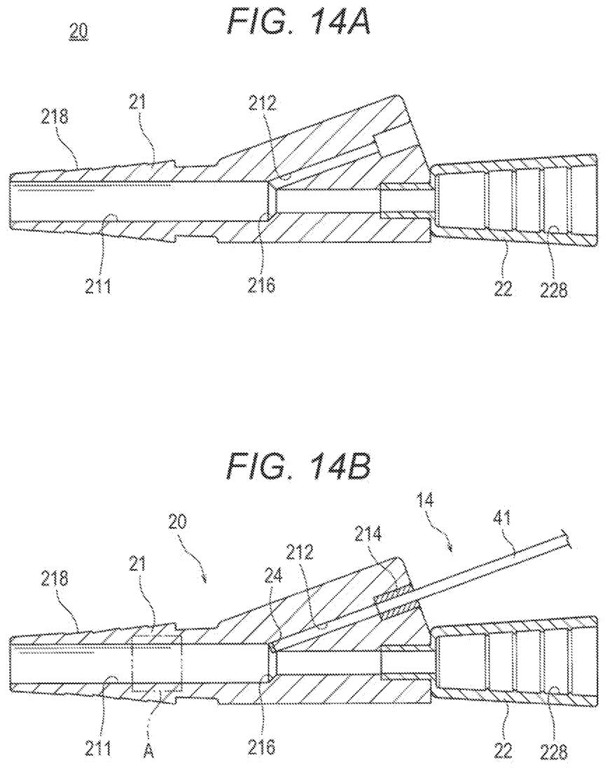

FIG. 14A is a cross-sectional view of the connection hub 20 according to Embodiment 2. The first conduit 211 has a large diameter portion on the catheter connection portion 218 side and a small diameter portion on the flexible hub member 22 side. A tapered portion 216 is provided at a boundary between the large diameter portion and the small diameter portion. The second conduit 212 is open on a tapered surface of the tapered portion 216. The holding tube 213 is not mounted to the second conduit 212.

FIG. 14B is an explanatory view for describing a method of using the connection hub 20 of Embodiment 2. In the present embodiment, the rubber holding member 214 is fixed to the optical fiber 41. A user can fix the optical fiber 41 by inserting the optical fiber 41 into the second conduit 212 and pushing the rubber holding member 214 into a stepped portion at an end of the second conduit 212. The rubber holding member 214 implements a function of a fixing portion of the present embodiment that is capable of fixing the measurement probe 14 in a state of being inserted into the second conduit 212. Further, a structure that serves as the rubber holding member 214 is not limited to rubber. It is also possible to directly fix the optical fiber 41 with an adhesive, or the optical fiber may be fixed by adopting a structure in which a metal member is crimped from the outside.

The light emitter 24 fixed to a distal end of the optical fiber 41 is disposed in the tapered portion 216 provided in the first conduit 211. By disposing the light emitter 24 at a place where a flow path of guided urine becomes relatively narrow, it is possible to provide the measurement system 10 in which the light emitter 24 reliably comes into contact with the urine.

A check valve that prevents backflow of urine may be provided in a portion indicated by A in FIG. 14B, that is, in the middle of the large diameter portion. By preventing backflow of the urine from the bag 171 into a bladder, a risk of occurrence of urinary tract infection can be reduced.

The holding tube 213, the rubber holding member 214, and the holding lid 215 may be provided at the end of the second conduit 212 as in Embodiment 1. The user may dispose the light emitter 24 at the tapered portion 216 according to a condition of a patient, or may dispose the light emitter 24 at a position closer to a distal end of the bladder indwelling catheter 15 than the tapered portion 216.

### Embodiment 3

The present embodiment relates to the measurement device 30 including a filter 57 that separates excitation light and fluorescence. Description of portions common to Embodiment 1 will be omitted.

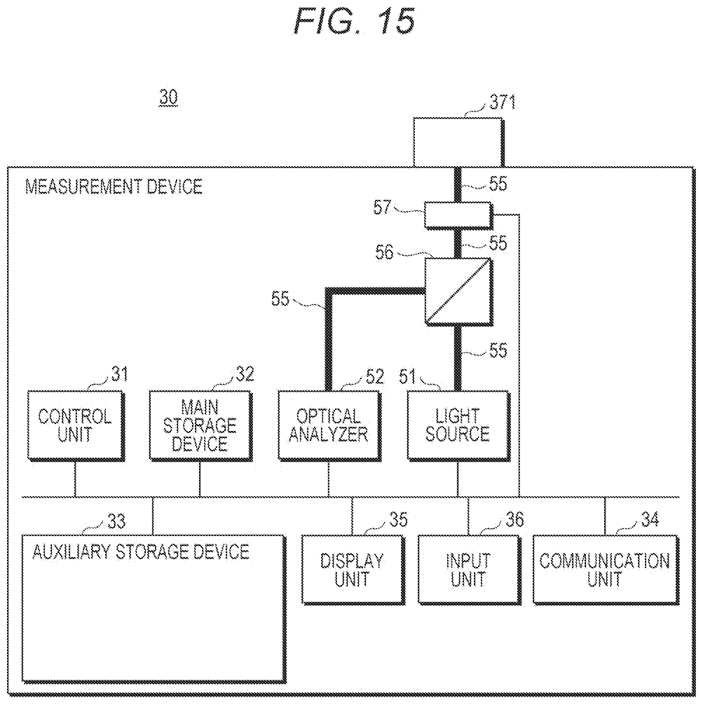

FIG. 15 is an explanatory view illustrating a configuration of the measurement device 30 of Embodiment 3. The filter 57 is disposed between the beam splitter 56 and the first connector 371 via the light guide path 55. The control unit 31 can adjust a wavelength range of light transmitted by the filter 57. The light source 51 according to the present embodiment emits broadband light including a wavelength of fluorescence in addition to a wavelength of excitation light. The light source 51 can be, for example, a white light-emitting diode (LED).

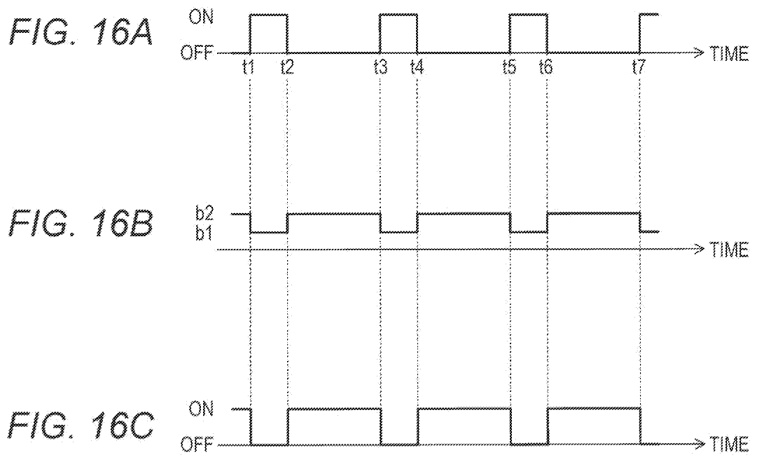

FIGS. 16A to 16C are time charts illustrating operation of the measurement device 30 of Embodiment 3. FIG. 16A illustrates ON and OFF timings of the light source 51. FIG. 16B illustrates operation timings of the filter 57. In FIG. 16B, b1 indicates that the filter 57 transmits excitation light. In FIG. 16B, b2 indicates that the filter 57 transmits fluorescence. FIG. 16C illustrates timings at which the optical analyzer 52 operates. ON indicates operation of analyzing characteristics of the fluorescence. OFF indicates a state in which the operation of analyzing characteristics of fluorescence is not performed. FIGS. 16A to 16C indicate time on horizontal axes.

During a period from time t1 to time t2, the light source 51 is turned ON. During this period, the filter 57 transmits excitation light. The optical analyzer 52 does not operate. The light emitter 24 is irradiated with the excitation light. In a case where the light emitter 24 is in contact with urine, fluorescence corresponding to a state of the urine is emitted.

During a period from time t2 to time t3, the light source 51 is turned OFF. During this period, the filter 57 transmits the fluorescence. The optical analyzer 52 analyzes characteristics of the fluorescence and outputs an oxygen partial pressure in the urine to the bus. The same operation is repeated after time t3.

According to the present embodiment, it is possible to provide the measurement system 10 capable of performing accurate measurement even in a case where the wavelength of the fluorescence is included in the light emitted by the light source 51.

### Embodiment 4

The present embodiment relates to the measurement system 10 capable of simultaneously measuring a plurality of items using one light source 51. Description of portions common to Embodiment 3 will be omitted.

The light emitter 24 according to the present embodiment includes two types of phosphors in a mixed manner. That is, two types of sensors are fixed to a distal portion of one bundle of the optical fibers 41 in the present embodiment. In the following description, the two types of phosphors are referred to as a phosphor J and a phosphor K. Wavelengths of fluorescence emitted from the phosphor J and the phosphor K are sufficiently separated from each other.

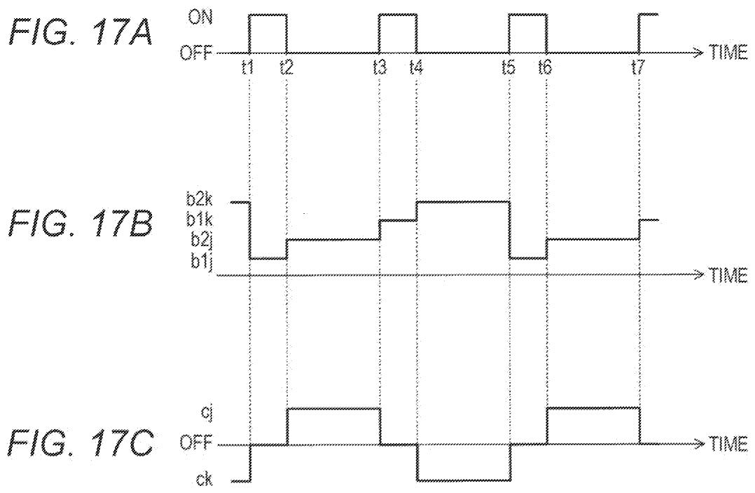

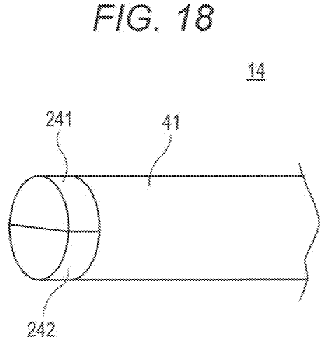

FIGS. 17A to 17C are time charts illustrating operation of the measurement device 30 of Embodiment 4. FIG. 17A illustrates ON and OFF timings of the light source 51. FIG. 17B illustrates operation timings of the filter 57. In FIG. 17B, b1j indicates that the filter 57 transmits excitation light of the phosphor J. In FIG. 17B, b2j indicates that the filter 57 transmits fluorescence emitted from the phosphor J. In FIG. 17B, b1k indicates that the filter 57 transmits excitation light of the phosphor K. In FIG. 17B, b2k indicates that the filter 57 transmits fluorescence emitted from the phosphor K.

FIG. 17C illustrates timings at which the optical analyzer 52 operates. In FIG. 17C, cj represents operation of analyzing characteristics of the fluorescence emitted from the phosphor J. In FIG. 17C, ck indicates operation of analyzing characteristics of the fluorescence emitted from the phosphor K. OFF indicates a state in which the operation of analyzing characteristics of fluorescence is not performed. FIGS. 17A to 17C indicate time on horizontal axes.

During a period from time t1 to time t2, the light source 51 is turned ON. During this period, the filter 57 transmits the excitation light of the phosphor J. The optical analyzer 52 does not operate. The light emitter 24 is irradiated with the excitation light. In a case where the light emitter 24 is in contact with urine, the phosphor J emits fluorescence corresponding to a state of the urine.

During a period from time t2 to time t3, the light source 51 is turned OFF. During this period, the filter 57 transmits the fluorescence emitted from the phosphor J. The optical analyzer 52 analyzes characteristics of the fluorescence and outputs items related to the phosphor J to the bus.

During a period from time t3 to time t4, the light source 51 is turned ON. During this period, the filter 57 transmits the excitation light of the phosphor K. The optical analyzer 52 does not operate. The light emitter 24 is irradiated with the excitation light. In a case where the light emitter 24 is in contact with urine, the phosphor K emits fluorescence corresponding to a state of the urine.

During a period from time t4 to time t5, the light source 51 is turned OFF. During this period, the filter 57 transmits the fluorescence emitted from the phosphor K. The optical analyzer 52 analyzes characteristics of the fluorescence and outputs items related to the phosphor K to the bus. The same operation is repeated after time t6.

According to the present embodiment, it is possible to provide the measurement system 10 capable of measuring the plurality of items using one light source 51. The light emitter 24 may have three or more kinds of phosphors. The filter 57 sequentially transmits excitation light and fluorescence of each of the phosphors.

### Modification 4-1

FIG. 18 is a perspective view of a distal portion of the measurement probe 14 of Modification 4-1. In the present modification, a first light emitter 241 mixed with the phosphor K and a second light emitter 242 mixed with the phosphor J are disposed on an end surface of the optical fiber 41.

FIG. 18 illustrates an example in which both the first light emitter 241 and the second light emitter 242 have a semicircular shape, but the first light emitter 241 and the second light emitter 242 may be disposed concentrically. A size of the first light emitter 241 and a size of the second light emitter 242 may be different from each other.

The measurement probe 14 illustrated in FIG. 18 may include the optical fiber connector 411 to which a fiber bundle that guides light emitted from the first light emitter 241 is connected and the optical fiber connector 411 to which a fiber bundle that guides light emitted from the second light emitter 242 is connected. The two optical fiber connectors 411 can be used by being connected to the separate measurement devices 30, respectively.

### Modification 4-2

The present modification relates to the measurement probe 14 in which the light emitters 24 are disposed at different positions along the longitudinal direction of the optical fiber 41.

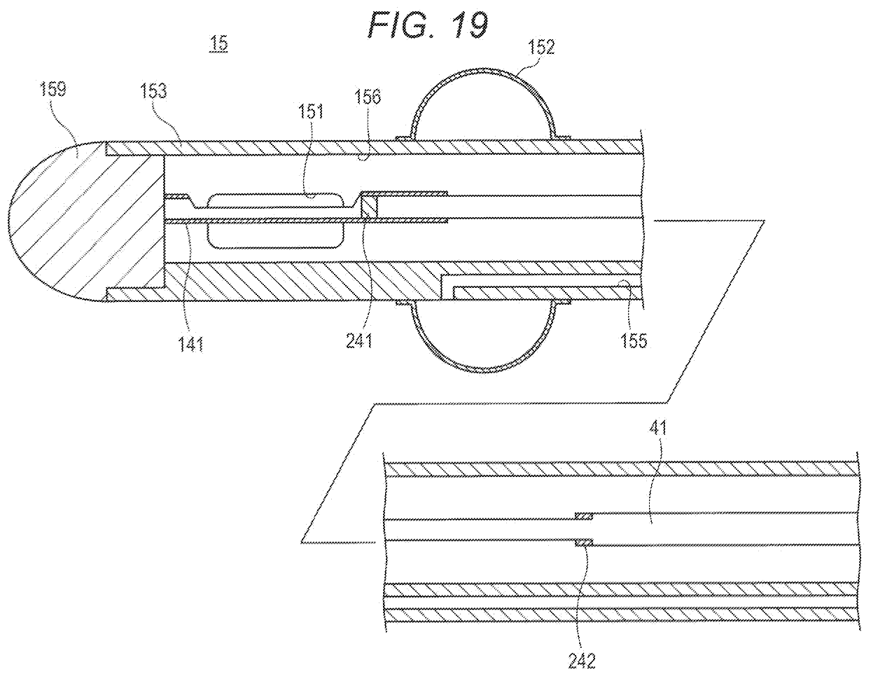

FIG. 19 is an enlarged cross-sectional view of a distal portion of the bladder indwelling catheter 15 into which the measurement probe 14 of Modification 4-2 is inserted. The first light emitter 241 is disposed on an end surface of the optical fiber 41, and the second light emitter 242 is disposed in the middle of the optical fiber 41. The second light emitter 242 has a ring shape surrounding the optical fiber 41 connected to the first light emitter 241.

### Modification 4-3

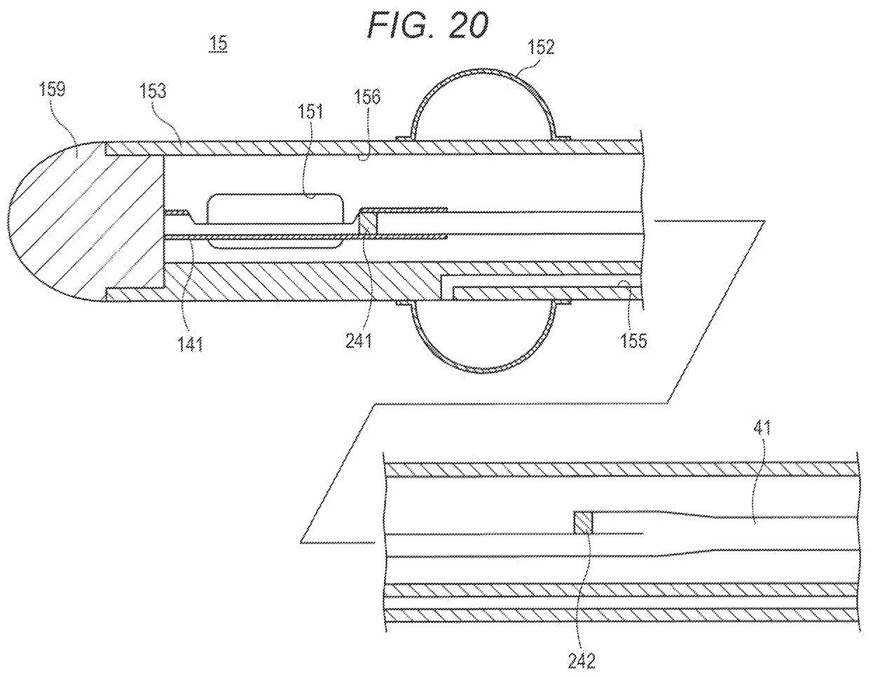

FIG. 20 is an enlarged cross-sectional view of a distal portion of the bladder indwelling catheter 15 into which the measurement probe 14 of Modification 4-3 is inserted. A short branch is provided in the middle of the optical fiber 41, and the second light emitter 242 is disposed on an end surface of the branch.

### Embodiment 5

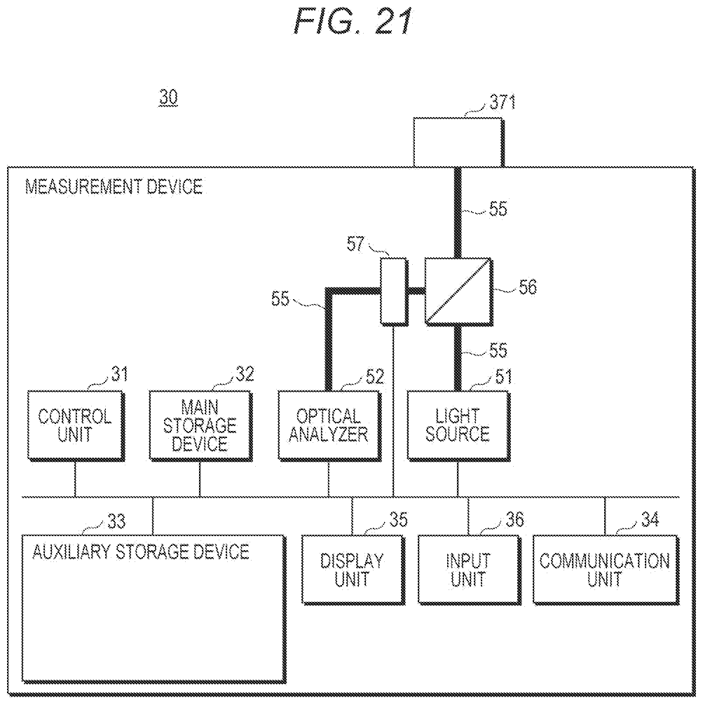

The present embodiment relates to the measurement system 10 using the light source 51 that switches light between a plurality of beams of narrow band excitation light and emits the light. Description of portions common to Embodiment 3 will be omitted. FIG. 21 is an explanatory view illustrating a configuration of the measurement device 30 of Embodiment 5. In the present embodiment, the filter 57 is disposed between the beam splitter 56 and the optical analyzer 52.

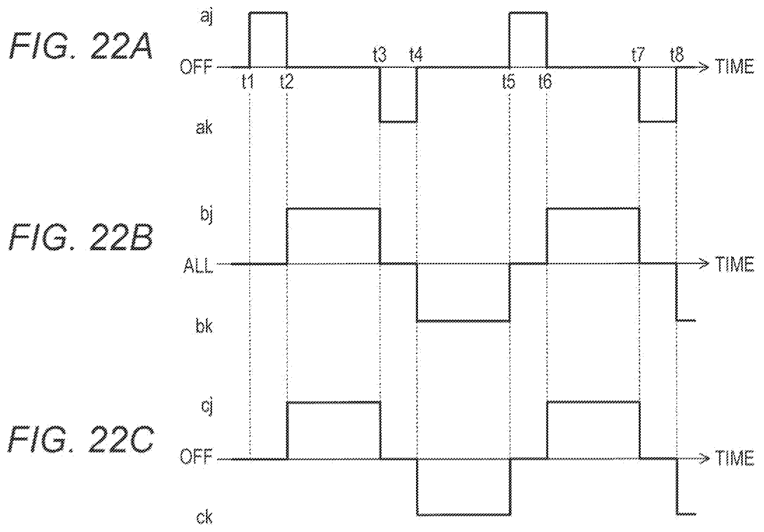

FIGS. 22A to 22C are time charts illustrating operation of the measurement device 30 of Embodiment 5. FIG. 22A illustrates timings at which the light source 51 operates. In FIG. 22A, aj indicates that the light source 51 emits excitation light of the phosphor J. In FIG. 22A, ak indicates that the light source 51 emits excitation light of the phosphor K.

FIG. 22B illustrates operation timings of the filter 57. ALL indicates that the filter 57 transmits all beams of light. In FIG. 22B, bj indicates that the filter 57 transmits fluorescence emitted from the phosphor J. In FIG. 22B, bk indicates that the filter 57 transmits fluorescence emitted from the phosphor K.

FIG. 22C illustrates timings at which the optical analyzer 52 operates. In FIG. 17C, cj represents operation of analyzing characteristics of the fluorescence emitted from the phosphor J. In FIG. 22C, ck represents operation of analyzing characteristics of the fluorescence emitted from the phosphor K. OFF indicates a state in which the operation of analyzing characteristics of fluorescence is not performed. FIGS. 22A to 22C indicate time on horizontal axes.

During a period from time t1 to time t2, the light source 51 emits excitation light for exciting the phosphor J. During this period, the filter 57 transmits all beams of light. The optical analyzer 52 does not operate. The light emitter 24 is irradiated with the excitation light. In a case where the light emitter 24 is in contact with urine, the phosphor J emits light according to a state of the urine.

During a period from time t2 to time t3, the light source 51 is turned OFF. During this period, the filter 57 transmits the fluorescence emitted from the phosphor J. The optical analyzer 52 analyzes characteristics of the fluorescence emitted from the phosphor J and outputs a result to the bus.

During a period from time t3 to time t4, the light source 51 emits excitation light for exciting the phosphor K. During this period, the filter 57 transmits all beams of light. The optical analyzer 52 does not operate. The light emitter 24 is irradiated with the excitation light. In a case where the light emitter 24 is in contact with urine, the phosphor K emits light according to a state of the urine.

During a period from time t4 to time t5, the light source 51 is turned OFF. During this period, the filter 57 transmits the fluorescence emitted from the phosphor K. The optical analyzer 52 analyzes characteristics of the fluorescence emitted from the phosphor K and outputs a result to the bus. The same operation is repeated after time t5.

According to the present embodiment, it is possible to provide the measurement system 10 capable of measuring a plurality of items using one light source 51 and one light emitter 24. The light emitter 24 may have three or more kinds of phosphors. The filter 57 sequentially switches a wavelength of light to be transmitted according to each of the phosphors.

The light source 51 may emit broadband light including both excitation light of the phosphor J and excitation light of the phosphor K. For example, the light source 51 may emit white light. In such a case, both aj and ak in FIG. 22A indicate that the light source 51 is turned ON.

### Embodiment 6

The present embodiment relates to the measurement device 30 including a plurality of the optical analyzers 52. Description of parts common to Embodiment 5 will be omitted.

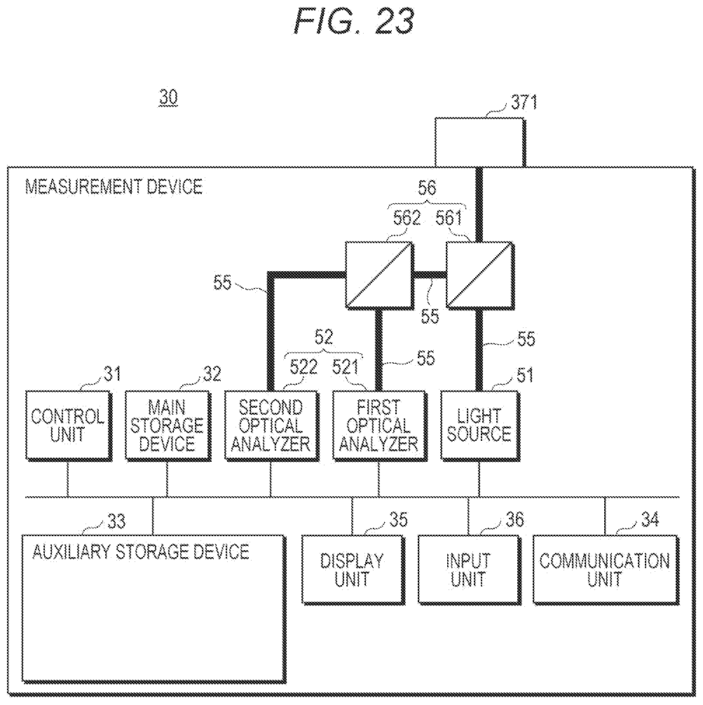

FIG. 23 is an explanatory view illustrating a configuration of the measurement device 30 of Embodiment 6. The measurement device 30 includes two optical analyzers 52 of a first optical analyzer 521 and a second optical analyzer 522, and two beam splitters 56 of a first beam splitter 561 and a second beam splitter 562.

The first beam splitter 561 is connected between the light source 51 and the first connector 371. The second beam splitter 562 is connected between the first beam splitter 561, and the first optical analyzer 521 and the second optical analyzer 522. The second beam splitter 562 is a dichroic beam splitter that separates incident light on the basis of a wavelength. The second beam splitter 562 implements a function of a spectroscopic unit that spectrally disperses fluorescence emitted from a plurality of phosphors.

In the following description, a case where the first optical analyzer 521 analyzes characteristics of fluorescence emitted from the phosphor J and the second optical analyzer 522 analyzes characteristics of fluorescence emitted from the phosphor K will be described as an example. Excitation light capable of exciting both the phosphor J and the phosphor K is emitted from the light source 51.

The phosphor J and the phosphor K can be mixed in one light emitter 24, for example. In a case where the measurement probe 14 includes the plurality of light emitters 24 of the first light emitter 241 and the second light emitter 242 as described with reference to FIGS. 18 to 20, one light emitter 24 may be mixed with the phosphor J, and the other light emitter 24 may be mixed with the phosphor K.

The excitation light irradiates the light emitter 24 via the light guide path 55, the beam splitter 56, and the optical fiber 41. In a case where the light emitter 24 comes into contact with urine flowing in the urine passage 156, the phosphor J and the phosphor K emits beams of the fluorescence, respectively.

Beams of the fluorescence emitted from the phosphor J and the phosphor K are incident on the optical fiber 41 in a mixed state. Beams of the fluorescence guided by the optical fiber 41 are incident on the first beam splitter 561 via the first connector 371 and the light guide path 55. Beams of the fluorescence are incident on the light guide path 55 connected to the second beam splitter 562 by the first beam splitter 561. Beams of the fluorescence are separated into the fluorescence emitted from the phosphor J and the other light by the second beam splitter 562. The fluorescence emitted from the phosphor J is incident on the first optical analyzer 521, and the other light is incident on the second optical analyzer 522.

The first optical analyzer 521 analyzes characteristics of the fluorescence emitted from the phosphor J and outputs a result to the bus. The second optical analyzer 522 analyzes characteristics of the fluorescence emitted from the phosphor K and outputs a result to the bus. An optical filter that transmits only the fluorescence emitted from the phosphor K may be disposed between the second beam splitter 562 and the second optical analyzer 522.

According to the present embodiment, it is possible to provide the measurement system 10 that simultaneously analyzes beams of the fluorescence emitted from the two types of phosphors. The light emitter 24 may include three or more types of phosphors, and the measurement device 30 may include the optical analyzers 52 and the beam splitters 56 as many as the number of phosphors. An optical filter that passes only a specific wavelength may be disposed in the middle of the light guide path 55 in order to adjust a wavelength of light passing through the light guide path 55 to an arbitrary wavelength.

### Embodiment 7

The present embodiment relates to the measurement system 10 using the bladder indwelling catheter 15 including a probe conduit 157 disposed in parallel with the urine passage 156. Description of portions common to Embodiment 1 will be omitted.

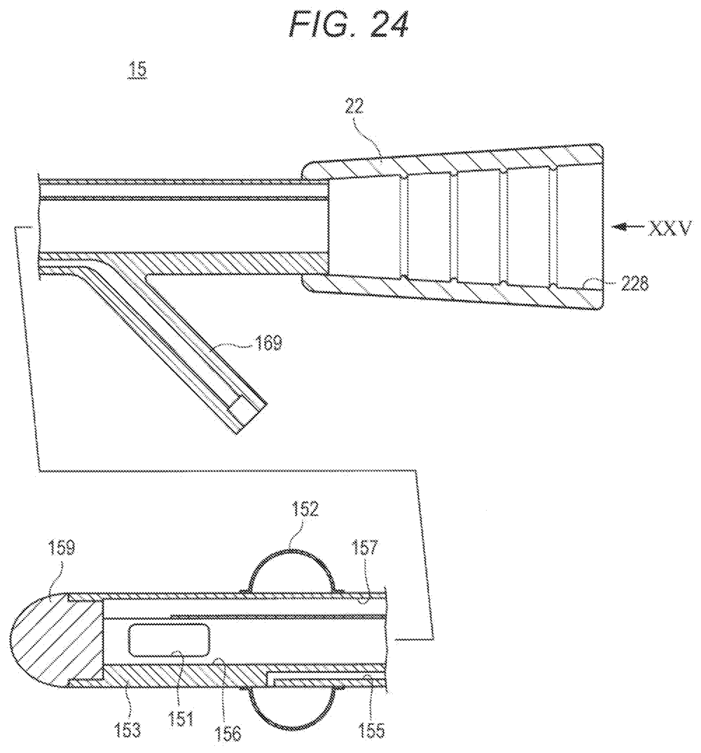

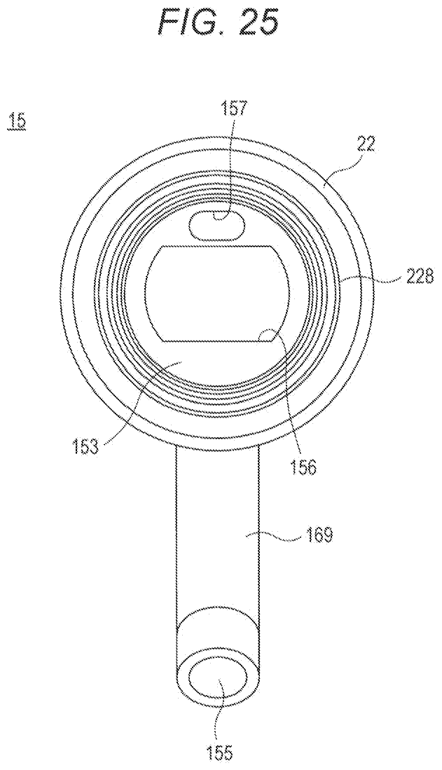

FIG. 24 is a cross-sectional view of the bladder indwelling catheter 15 of Embodiment 7. FIG. 25 is a view taken along arrow XXV in FIG. 24. The shaft 153 of the present embodiment is a so-called multi-lumen tube having three conduits of the urine passage 156, the balloon conduit 155, and the probe conduit 157.

The probe conduit 157 communicates with the urine passage 156 in the middle of the bladder indwelling catheter 15. The urine passage 156 and the probe conduit 157 are open on an end surface of the shaft 153 on the flexible hub member 22 side.

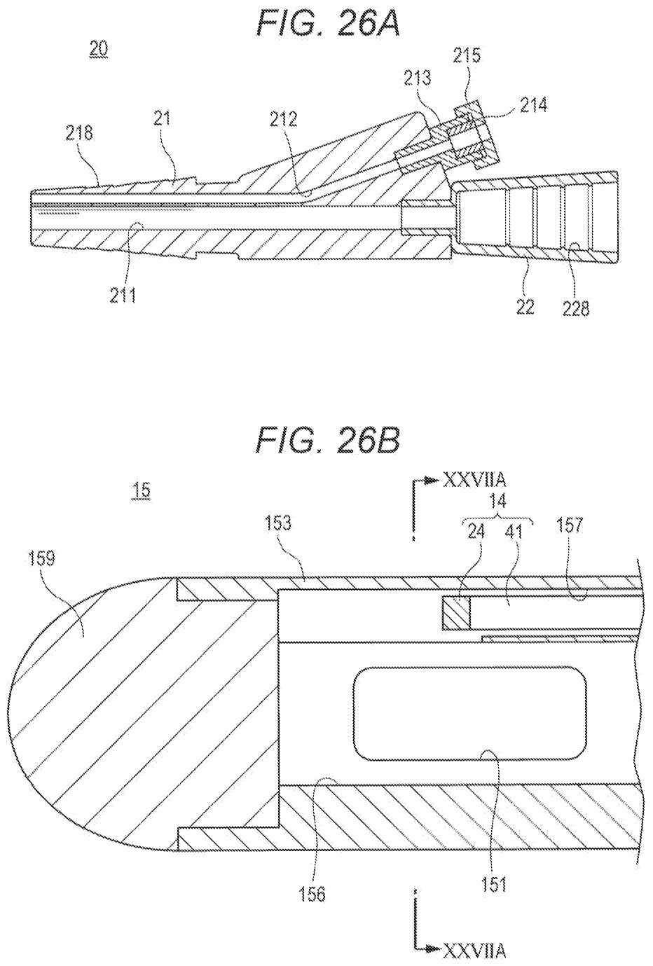

FIG. 26A is a cross-sectional view of the connection hub 20 of Embodiment 7. Two independent conduits of the first conduit 211 and the second conduit 212 are provided in the connection hub 20. That is, the first conduit 211 and the second conduit 212 do not join in the present embodiment.

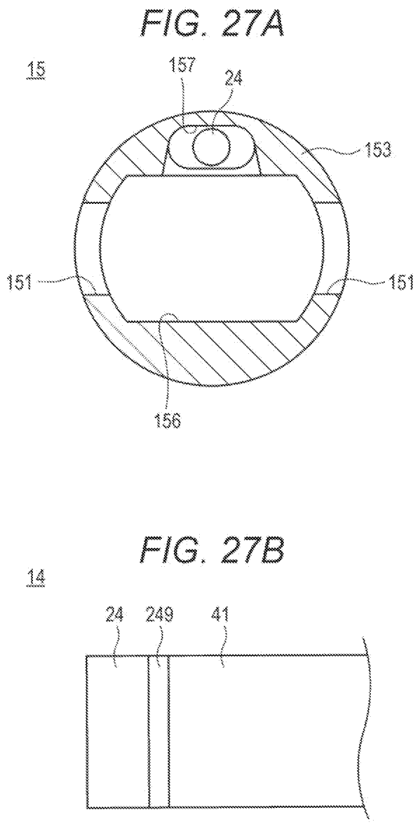

FIG. 26B is an enlarged cross-sectional view of a distal portion of the bladder indwelling catheter 15 into which the measurement probe 14 of Embodiment 7 is inserted. FIG. 27A is a cross-sectional view taken along line XXVIIA-XXVIIA in FIG. 26B.

As illustrated in FIG. 26B, a partition wall between the probe conduit 157 and the urine passage 156 is removed in a range from a distal end side of the bladder indwelling catheter 15 to the middle of the side hole 151. The measurement probe 14 is inserted through the probe conduit 157. The light emitter 24 provided at a distal end of the measurement probe 14 is located near the side hole 151. The rubber holding member 214 implements a function of a fixing portion of the present embodiment that is capable of fixing the measurement probe 14 in a state of being inserted into the probe conduit 157.

According to the present embodiment, the measurement probe 14 is inserted into the probe conduit 157 having a smaller diameter than the urine passage 156. Thus, even in a case where a shape of the shaft 153 has changed, positions of the distal end of the measurement probe 14 and the distal end of the bladder indwelling catheter 15 change relatively little. Thus, it is possible to provide the measurement system 10 in which the measurement probe 14 is less likely to be pressed against the distal end side of the bladder indwelling catheter 15 even when a posture of a patient changes.

A sensor holding portion such as the housing 141, the coil 142, the cover plate 143, or the wire 144 described in Embodiment 1 and its modifications may be provided at the distal end of the measurement probe 14, and may abut against the distal end member 159.

The partition wall between the probe conduit 157 and the urine passage 156 may be removed up to the urination funnel 154 side further from the side hole 151. In such a case, the light emitter 24 can be disposed on the urination funnel 154 side with respect to the side holes 151. It is possible to provide the measurement system 10 capable of performing more accurate measurement as fresh urine reliably touches the light emitter 24.

The measurement probe 14 and the bladder indwelling catheter 15 are desirably single use products supplied to a user in a sterilized state. A catheter set in which the measurement probe 14 is combined with the bladder indwelling catheter 15 may be supplied to the user.

### Modification 7-1

FIG. 27B is an enlarged view of a distal portion of the measurement probe 14 of Modification 7-1. In the present modification, the light emitter 24 formed in a sheet shape is fixed to an end surface of the optical fiber 41 via an adhesive layer 249.

The light emitter 24 can be, for example, a plate made of a translucent resin into which a phosphor is mixed. The light emitter 24 may be a translucent plate coated with a phosphor. When an adhesive having a short curing time is used for the adhesive layer 249, the measurement probe 14 that can be manufactured in a relatively short time can be provided. Further, a layer for preventing degradation of a dye due to ambient light may be formed on a surface of the light emitter 24. By forming a layer containing carbon black or the like on the surface of the light emitter 24 as a coating or separately in a plate shape, it is possible to prevent the light emitter 24 from being exposed to unnecessary light.

### Modification 7-2

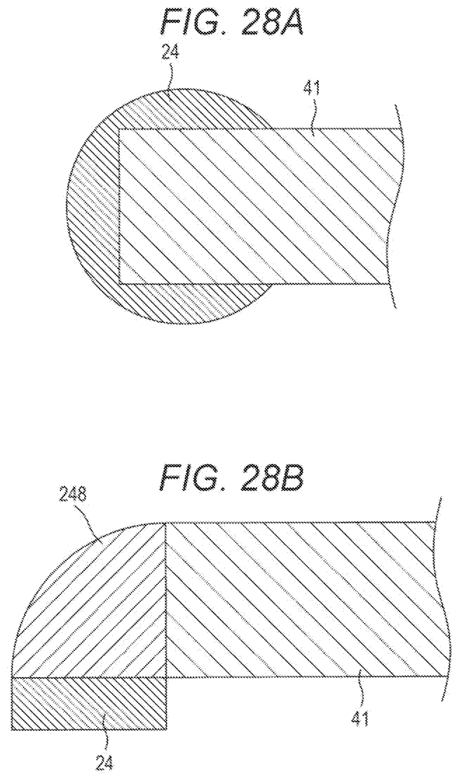

FIG. 28A is an enlarged cross-sectional view of a distal portion of the measurement probe 14 of Modification 7-2. In the present modification, an end of the optical fiber 41 is covered with the light emitter 24.

For example, a distal end of the optical fiber 41 is immersed in an uncured transparent resin into which a phosphor has been mixed, is pulled up, and then, is cured, whereby the optical fiber 41 of the present modification can be manufactured. A transparent resin into which a phosphor is mixed may be molded at a distal end of the optical fiber 41 using a mold.

### Modification 7-3

FIG. 28B is an enlarged cross-sectional view of a distal portion of the measurement probe 14 of Modification 7-3. In the present modification, the plate-shaped light emitter 24 is fixed substantially perpendicularly to an end surface of the optical fiber 41.

A light guide portion 248 made of, for example, a translucent resin is disposed between an end of the optical fiber 41 and the light emitter 24. The optical fiber 41 and the light emitter 24 are attached and fixed to the light guide portion 248 by an adhesive layer. The light guide portion 248 may also serve as an adhesive layer for attaching and fixing the optical fiber 41 and the light emitter 24. Further, the light guide portion 248 may be formed into a shape in FIG. 28B by polishing or molding the optical fiber 41.

### Modification 7-4

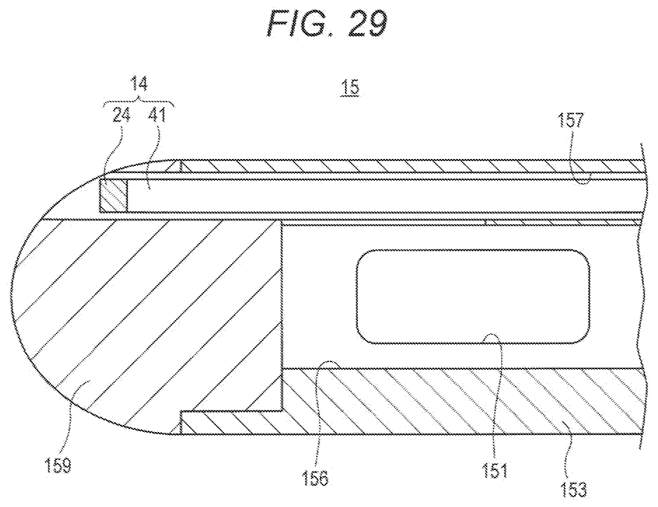

FIG. 29 is an enlarged cross-sectional view of a distal portion of the bladder indwelling catheter 15 into which the measurement probe 14 of Modification 7-4 is inserted. In the present modification, a through-hole provided in the distal end member 159 communicates with the probe conduit 157. The light emitter 24 is disposed near the distal end member 159 and can come into contact with fresh urine in a bladder. By defining an insertion length at a hand-side portion into which the optical fiber 41 is inserted, it is possible to help prevent a protrusion from a distal end of the catheter. Positioning is performed assuming jumping-out when the catheter is bent.

In FIG. 29, since the probe conduit 157 and the shaft 153 communicate with each other near the distal end of the bladder indwelling catheter 15, the urine in the bladder flows into the urine passage 156 through a gap between the probe conduit 157 and the measurement probe 14 in addition to the side hole 151. Thus, the light emitter 24 is easily touched by fresh urine.

### Modification 7-5

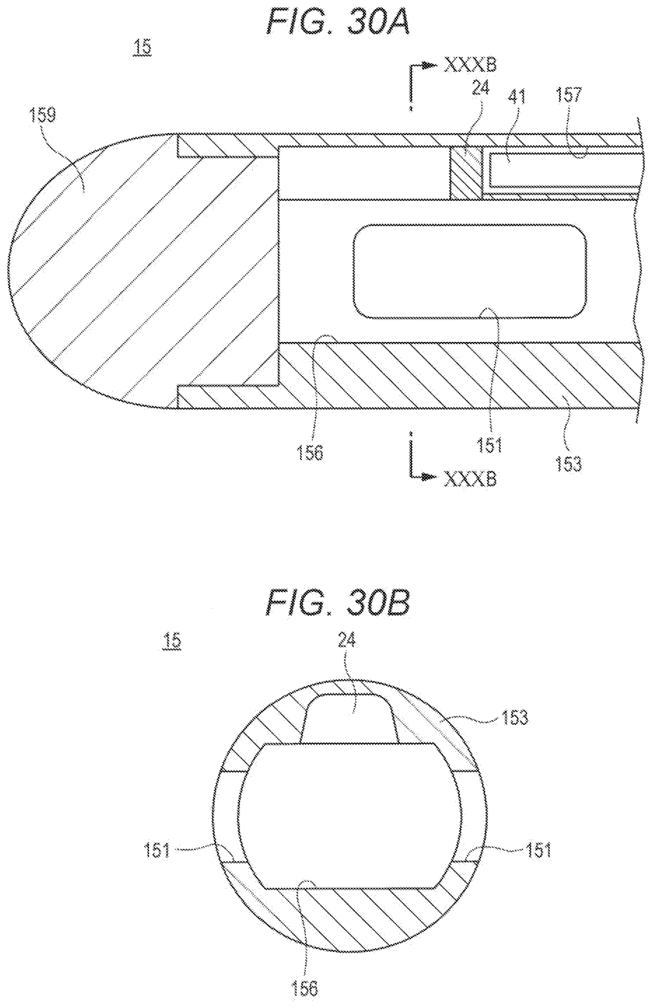

FIG. 30A is an enlarged cross-sectional view of a distal portion of the bladder indwelling catheter 15 into which the measurement probe 14 of Modification 7-5 is inserted. FIG. 30B is a cross-sectional view taken along line XXXB-XXXB in FIG. 30A. In the present modification, a distal portion of the probe conduit 157 is sealed with the light emitter 24. The measurement probe 14 inserted into the probe conduit 157 does not have the light emitter 24 at an end of the optical fiber 41.

The light emitter 24 comes into contact with a quencher contained in urine flowing in from the side hole 151 and emits fluorescence. The fluorescence is propagated to the measurement device 30 through the optical fiber 41. An end surface of the optical fiber 41 may abut against the light emitter 24 or may have a gap with respect to the light emitter 24. In order to obtain optical stability, an optical lens made of a flexible resin may be present between the light emitter 24 and the optical fiber 41.

### Modification 7-6

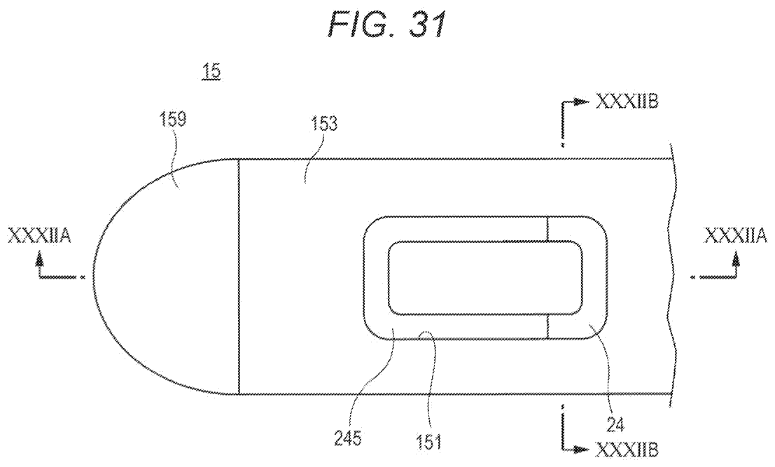

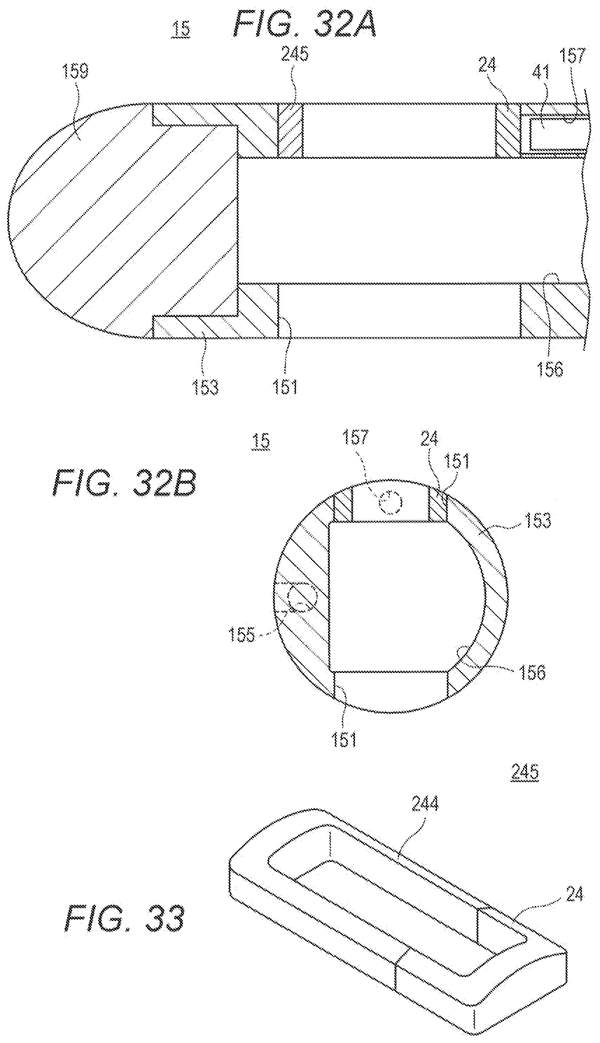

FIG. 31 is a front view of a distal portion of the bladder indwelling catheter 15 into which the measurement probe 14 of Modification 7-6 is inserted. FIG. 32A is a cross-sectional view taken along line XXXIIA-XXXIIA in FIG. 31. FIG. 32B is a cross-sectional view taken along line XXXIIB-XXXIIB in FIG. 31.

In the present modification, a light emitter ring 245 is fitted into an inner surface of the side hole 151. FIG. 33 is a perspective view of the light emitter ring 245. The light emitter ring 245 has a ring shape in which a through-hole is formed in a middle high portion of a substantially semi-cylindrical shape. The light emitter ring 245 includes the light emitter 24 and a non-light emitting portion 244. The light emitter 24 can be, for example, a translucent resin into which a phosphor is mixed. The non-light emitting portion 244 is the same translucent resin as a base material of the light emitter 24.

As illustrated in FIGS. 31 and 32A, the light emitter ring 245 is mounted such that the light emitter 24 is on the urination funnel 154 side. As illustrated in FIG. 32B, the light emitter 24 blocks an end of the probe conduit 157.

The light emitter 24 comes into contact with a quencher contained in urine and emits fluorescence. The fluorescence is propagated to the measurement device 30 through the optical fiber 41. An end surface of the optical fiber 41 may abut against the light emitter 24 or may have a gap with respect to the light emitter 24.

According to the present modification, the light emitter ring 245 and the shaft 153 can be attached over the entire circumference of the inner surface of the side hole 151, and thus, a relatively strong adhesion can be achieved. Since the same resin material is used for the base material of the light emitter 24 and the non-light emitting portion 244, detachment between the light emitter 24 and the non-light emitting portion 244 can be prevented. The entire light emitter ring 245 may be formed of the light emitter 24.

### Modification 7-7

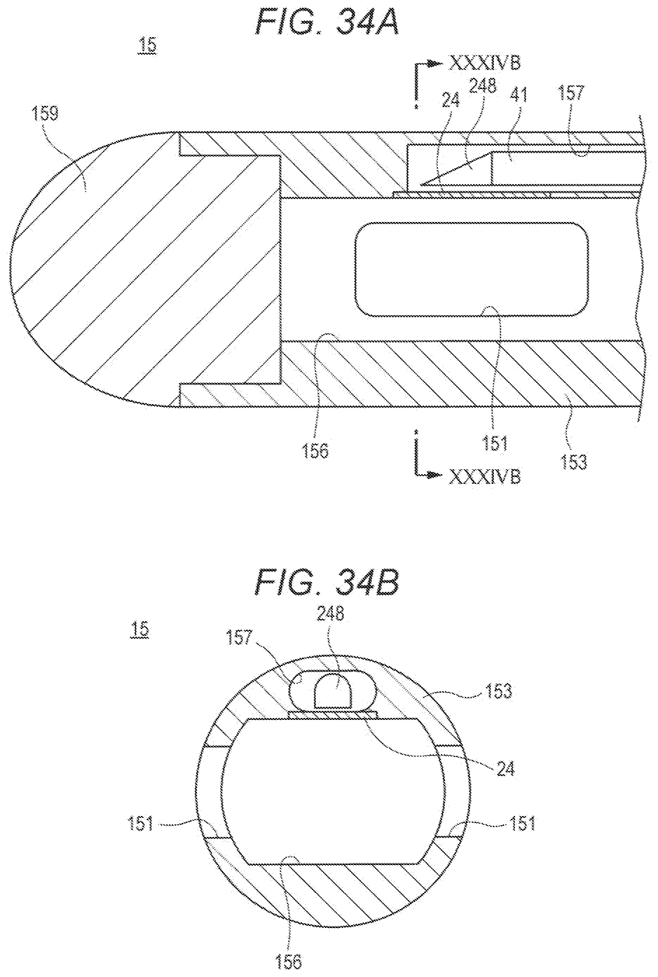

FIG. 34A is an enlarged cross-sectional view of a distal portion of the bladder indwelling catheter 15 into which the measurement probe 14 of Modification 7-7 is inserted. FIG. 34B is a cross-sectional view taken along line XXXIVB-XXXIVB in FIG. 34A.

In the present modification, the light emitter 24 having a plate shape is disposed between the urine passage 156 and the probe conduit 157. The light guide portion 248 having a substantially prism shape is mounted to an end of the optical fiber 41.

The light emitter 24 touches urine that has entered the urine passage 156 through the side hole 151 and emits fluorescence. The fluorescence is guided to the optical fiber 41 by the light guide portion 248. According to the present modification, the fluorescence emitted from the light emitter 24 having a larger area than an end surface of the optical fiber 41 is guided to the optical fiber 41 by the light guide portion 248, so that the highly sensitive measurement system 10 can be provided.

### Modification 7-8

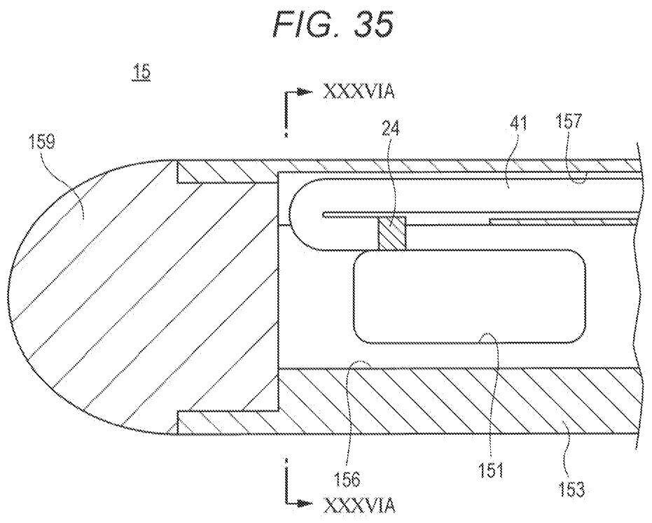

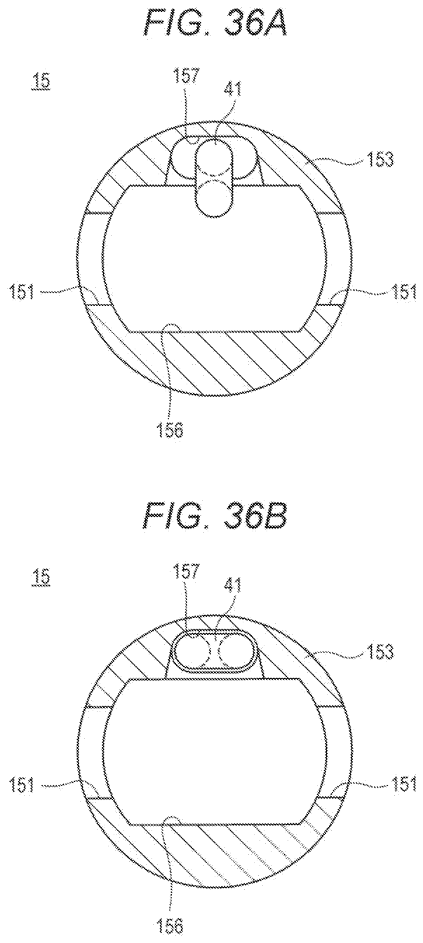

FIG. 35 is an enlarged cross-sectional view of a distal portion of the bladder indwelling catheter 15 into which the measurement probe 14 of Modification 7-8 is inserted. FIG. 36A is a cross-sectional view taken along line XXXVIA-XXXVIA in FIG. 35. The optical fiber 41 of the present modification is molded in a state where a distal end is folded (or bent over on itself), and has an end surface on which the light emitter 24 is disposed.

FIG. 36B is an explanatory view illustrating a method of inserting the measurement probe 14 of Modification 7-8 into the probe conduit 157. The measurement probe 14 is inserted into the probe conduit 157 in a state where a longitudinal axis of the probe conduit 157 having an oval cross section is set to coincide with a folding direction of the optical fiber 41. After a folded portion of the measurement probe 14 is inserted until abutting against the distal end member 159, the measurement probe 14 is rotated by approximately 90 degrees to achieve a state illustrated in FIGS. 35 and 36A.

According to the present modification, the light emitter 24 of the measurement probe 14 inserted into the probe conduit 157 having a small diameter can be disposed in the urine passage 156. Thus, the measurement system 10 in which the light emitter 24 is touched by fresh urine can be provided.

### Modification 7-9

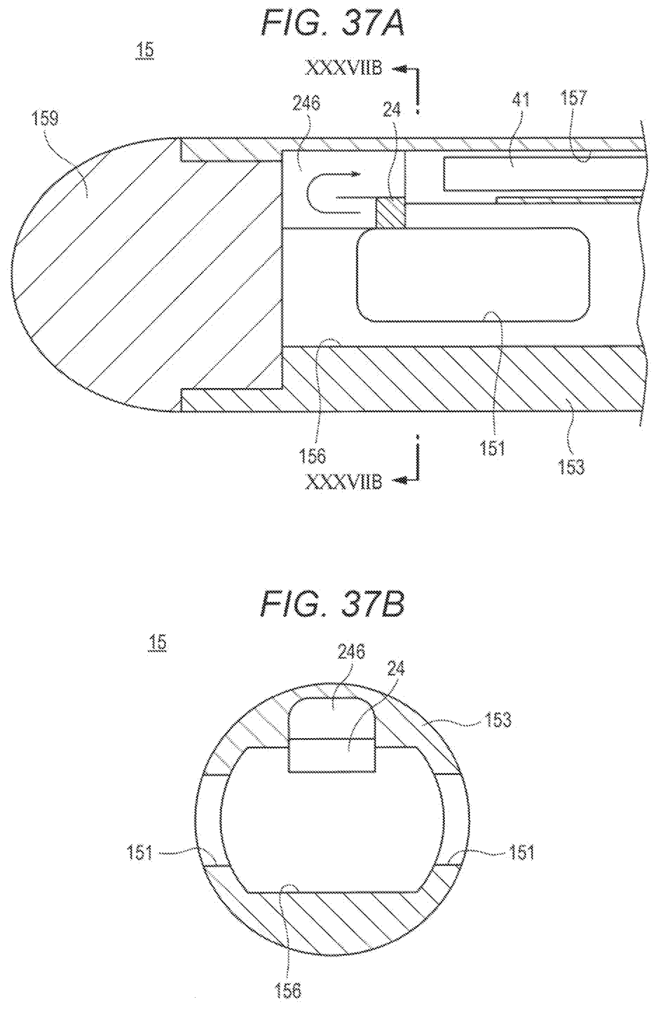

FIG. 37A is an enlarged cross-sectional view of a distal portion of the bladder indwelling catheter 15 into which the measurement probe 14 of Modification 7-9 is inserted. FIG. 37B is a cross-sectional view taken along line XXXVIIB-XXXVIIB in FIG. 37A. In the present modification, a light emitter block 246 is disposed on an inner surface of the distal portion of the bladder indwelling catheter 15. The light emitter 24 is disposed on a surface of the light emitter block 246. Fluorescence emitted from the light emitter 24 due to contact with urine is guided in a substantially U shape as indicated by an arrow in FIG. 37A and is incident on an end surface of the optical fiber 41.

The light emitter block 246 can be formed by, for example, bending an optical fiber bundle into a U shape. The light emitter block 246 may be a combination of a plurality of optical parts such as a light guide and a prism.

### Modification 7-10

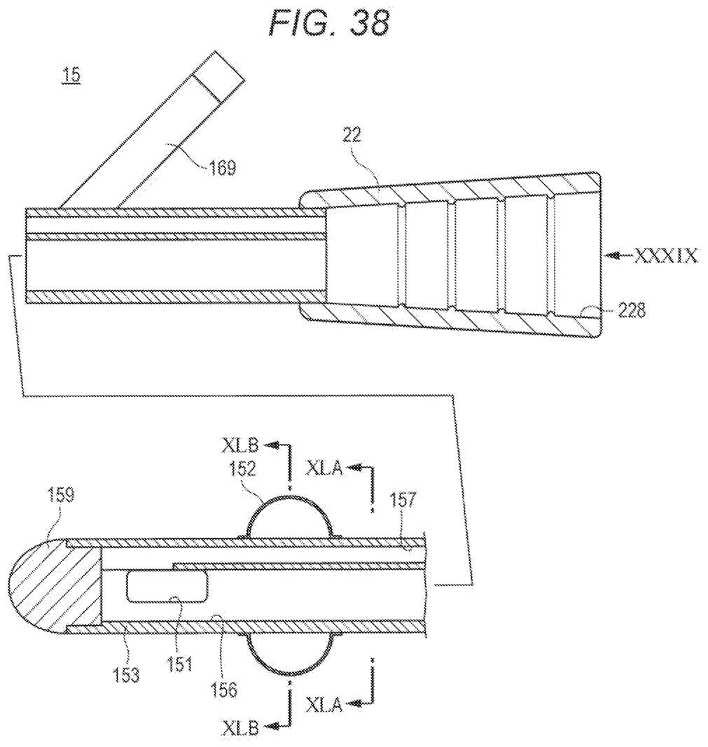

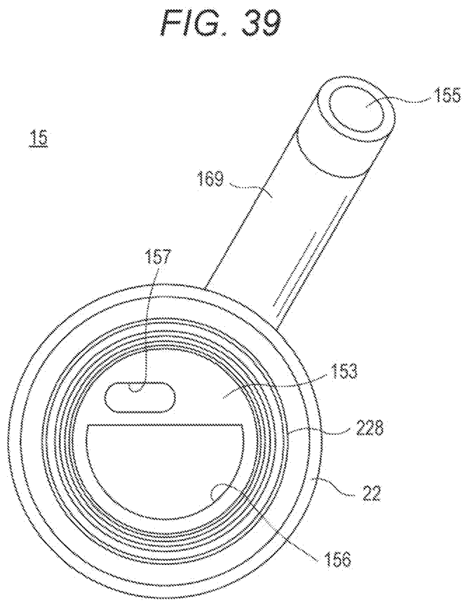

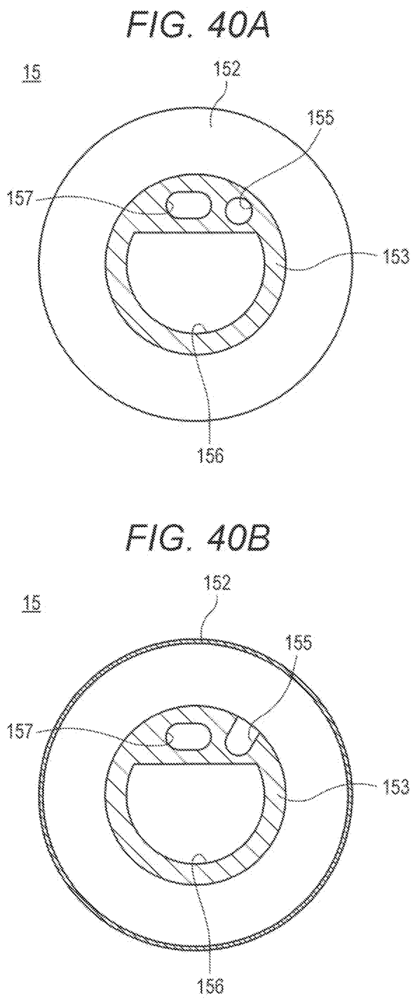

FIG. 38 is a cross-sectional view of the bladder indwelling catheter 15 of Modification 7-10. FIG. 39 is a view taken along arrow XXXIX in FIG. 38. FIG. 40A is a cross-sectional view taken along line XLA-XLA in FIG. 38. FIG. 40B is a cross-sectional view taken along line XLB-XLB in FIG. 38.

In the present modification, the shaft 153 is a multi-lumen tube having three conduits of the balloon conduit 155, the urine passage 156, and the probe conduit 157. The urine passage 156 has a substantially semi-cylindrical cross section. The balloon conduit 155 and the probe conduit 157 are disposed in parallel on the upper side in FIGS. 38 to 40B. As illustrated in FIG. 40B, the balloon conduit 155 is open to the inner side of the balloon 152.

### Modification 7-11

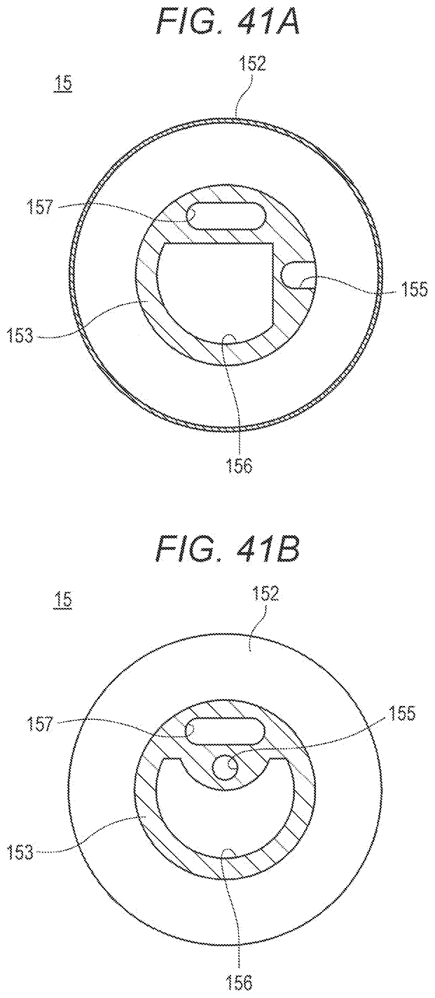

FIG. 41A is a cross-sectional view of the bladder indwelling catheter 15 of Modification 7-11. FIG. 41A illustrates a cross section at a position similar to that of FIG. 40B. In the present modification, the shaft 153 is a multi-lumen tube having three conduits of the balloon conduit 155, the urine passage 156, and the probe conduit 157.

In the present modification, the urine passage 156 has a substantially fan shape. The probe conduit 157 and the balloon conduit 155 are disposed near two sides of the urine passage 156, respectively. The balloon conduit 155 is open to the inner side of the balloon 152.

### Modification 7-12

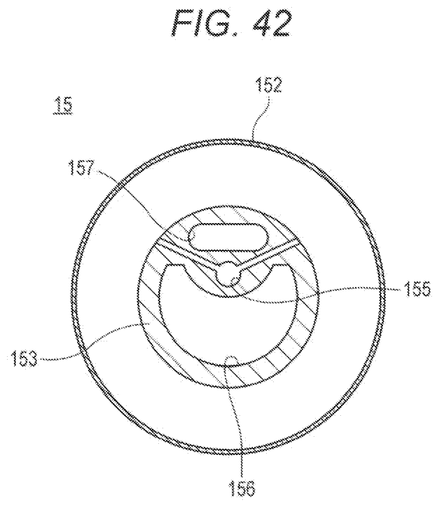

FIGS. 41B and 42 are cross-sectional views of the bladder indwelling catheter 15 of Modification 7-12. FIG. 41B illustrates a cross section at a position similar to that of FIG. 40A. FIG. 42 illustrates a cross section at a position similar to that of FIG. 40B. In the present modification, the shaft 153 is a multi-lumen tube having three conduits of the balloon conduit 155, the urine passage 156, and the probe conduit 157.

In the present modification, the urine passage 156 has a substantially crescent-shaped cross section. The balloon conduit 155 is disposed in a depression portion of the crescent shape. The probe conduit 157 having an oval cross section is disposed on the opposite side of the urine passage 156 across the balloon conduit 155. As illustrated in FIG. 42, two communication paths connecting the balloon conduit 155 and the inside of the balloon 152 are provided.

### Modification 7-13

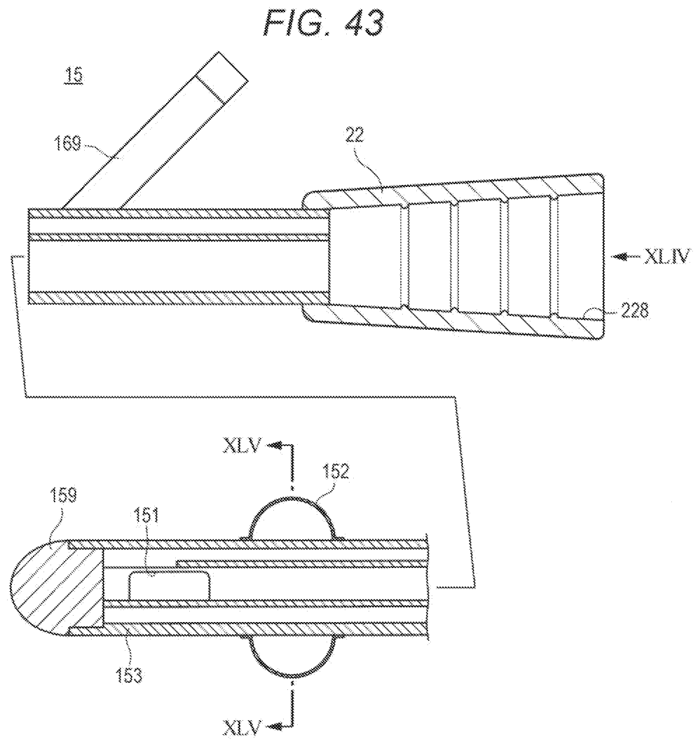

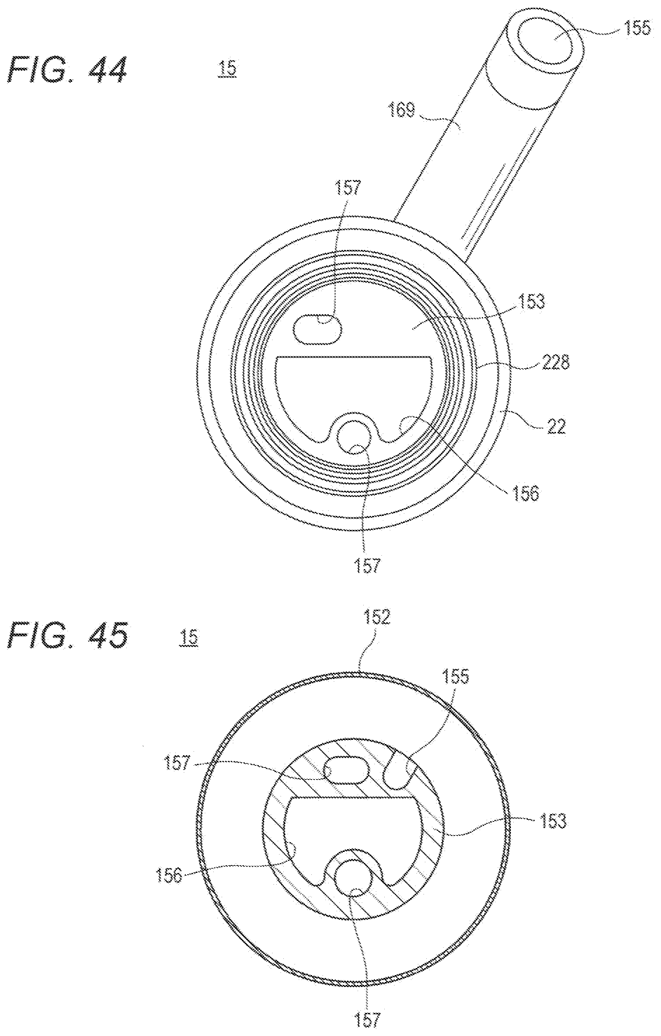

FIG. 43 is a cross-sectional view of the bladder indwelling catheter 15 of Modification 7-13. FIG. 44 is a view taken along arrow XLIV in FIG. 43. FIG. 45 is a cross-sectional view taken along line XLV-XLV in FIG. 43. In the present modification, the shaft 153 is a multi-lumen tube having a total of four conduits including the urine passage 156 and two probe conduits 157.

In the present modification, the urine passage 156 has a substantially concave cross section with a depression facing downward in FIG. 45. One probe conduit 157 is disposed in the depression of a concave portion. The other probe conduit 157 having a substantially oval cross section and the balloon conduit 155 are disposed above the urine passage 156 in FIG. 45. As illustrated in FIG. 44, the balloon conduit 155 is open to the balloon water injection portion 169 on the flexible hub member 22 side.

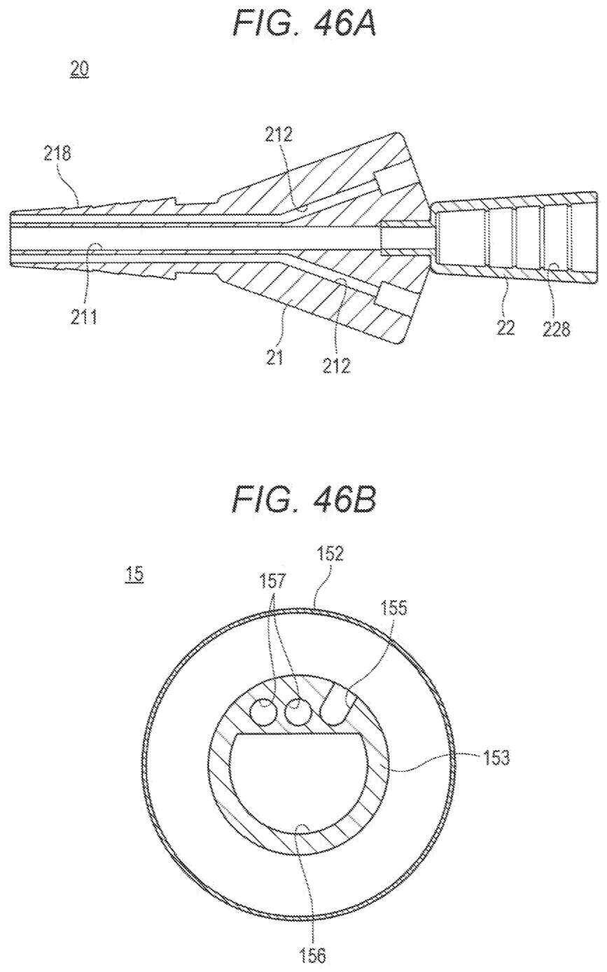

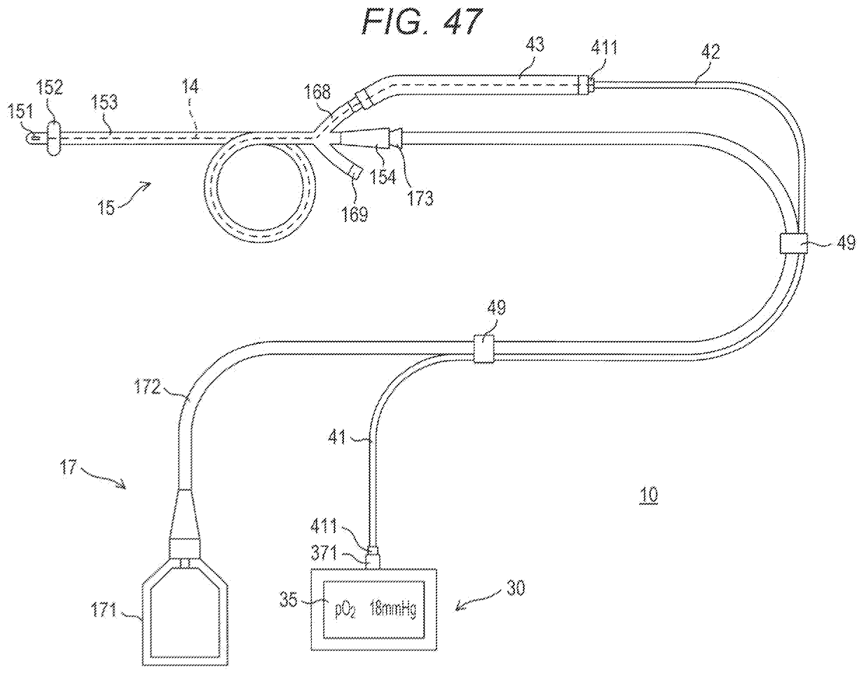

FIG. 46A is a cross-sectional view of the connection hub 20 of Modification 7-13. The connection hub 20 of the present modification includes two independent second conduits 212. The first conduit 211 and the two second conduits 212 are disposed so as to communicate with the urine passage 156 and the two probe conduits 157, respectively, when the bladder indwelling catheter 15 and the connection hub 20 are connected.

According to the present modification, it is possible to provide the measurement system 10 capable of simultaneously using the two measurement probes 14. The two measurement probes 14 are used by being connected to the separate measurement devices 30, respectively. The measurement device 30 may be capable of being simultaneously connected to the two measurement probes 14.

One probe conduit 157 does not necessarily communicate with the urine passage 156. It is possible to perform measurement by inserting a sensor, which performs measurement in a non-wetted manner, such as a sensor of a laser flowmeter, into the conduit not communicating with the urine passage 156. It is possible to provide the measurement system 10 that can simultaneously use a sensor that performs measurement in a wetted manner using the light emitter 24 and the sensor that performs measurement in a non-wetted manner.

The probe conduit 157 not communicating with the urine passage 156 is an example of a non-communicating conduit of the present modification. The sensor of the laser flowmeter is an example of a non-wetted sensor of the present modification.

### Modification 7-14

FIG. 46B is a cross-sectional view of the bladder indwelling catheter 15 of Modification 7-14. FIG. 46B illustrates a cross section at a position similar to that of FIG. 45. In the present modification, the shaft 153 is a multi-lumen tube having a total of four conduits including the urine passage 156 and two probe conduits 157.

In the present modification, the urine passage 156 has a substantially semi-cylindrical cross section having a linear portion disposed on the upper side in FIG. 46B. The balloon conduit 155 and the two probe conduits 157 are disposed above the urine passage 156.

### Embodiment 8

The present embodiment relates to the measurement system 10 including an adapter 43. Description of portions common to Embodiment 1 will be omitted.

FIG. 47 is an explanatory view illustrating a configuration of the measurement system 10 of Embodiment 8. In the present embodiment, the bladder indwelling catheter 15 and the urine collection bag 17 are directly connected without the intervention of the connection hub 20. The bladder indwelling catheter 15 is a so-called 3-way type including the balloon water injection portion 169 and a probe port 168. The adapter 43 is mounted to the probe port 168. The measurement probe 14 is inserted through the adapter 43 and the bladder indwelling catheter 15.

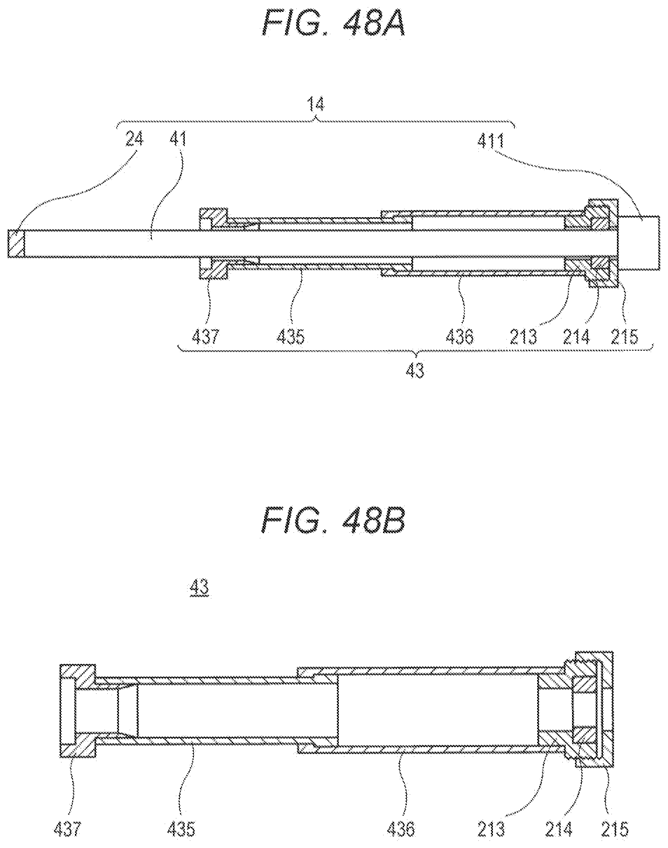

FIG. 48A is a cross-sectional view of the measurement probe 14 to which the adapter 43 is mounted. FIG. 48B is a cross-sectional view of the adapter 43. FIGS. 48A and 48B are views schematically illustrating configurations of the measurement probe 14 and the adapter 43 by reducing a longitudinal dimension of the measurement probe 14.

The measurement probe 14 includes the optical fiber 41, the light emitter 24 disposed on one end surface of the optical fiber 41, and the optical fiber connector 411 connected to the other end of the optical fiber 41.

As illustrated in FIG. 48B, the adapter 43 includes an inner tube 435, an outer tube 436, a mounting portion 437, the holding tube 213, the rubber holding member 214, and the holding lid 215. The mounting portion 437 connectable to the probe port 168 is fixed to one end of the inner tube 435. A fixing structure between the probe port 168 and the outer tube 436 can be, for example, a luer lock structure.

The inner tube 435 and the outer tube 436 are slidable, and retainers (i.e., retaining structure) are provided at both ends of the inner tube 435 and the outer tube 436. The holding tube 213 holding the rubber holding member 214 is fixed to the end of the outer tube 436. The holding lid 215 is mounted to the holding tube 213. Structures of the holding tube 213, the rubber holding member 214, and the holding lid 215 are similar to those in Embodiment 1.

Each of the inner tube 435 and the outer tube 436 can be a tube made of a resin having flexibility. For a retaining structure between the inner tube 435 and the outer tube 436, for example, a separate part made of hard plastic or metal may be used. The retaining structure between the inner tube 435 and the outer tube 436 desirably has a lock mechanism such that the inner tube 435 can be fixed in a state of being pulled out from the outer tube 436.

As illustrated in FIG. 48A, the measurement probe 14 is inserted through the adapter 43. The optical fiber 41 is fixed by the holding lid 215 and the rubber holding member 214. When the holding lid 215 is tightened at a position where the optical fiber connector 411 is in contact with the holding lid 215, a distal end of the optical fiber 41 protrudes from the inner tube 435.

For example, the measurement probe 14 is supplied to a user in a state of being inserted into the adapter 43 as illustrated in FIG. 48A. Note that the measurement probe 14 and the adapter 43 may be separately supplied and assembled by the user into the state illustrated in FIG. 48A.

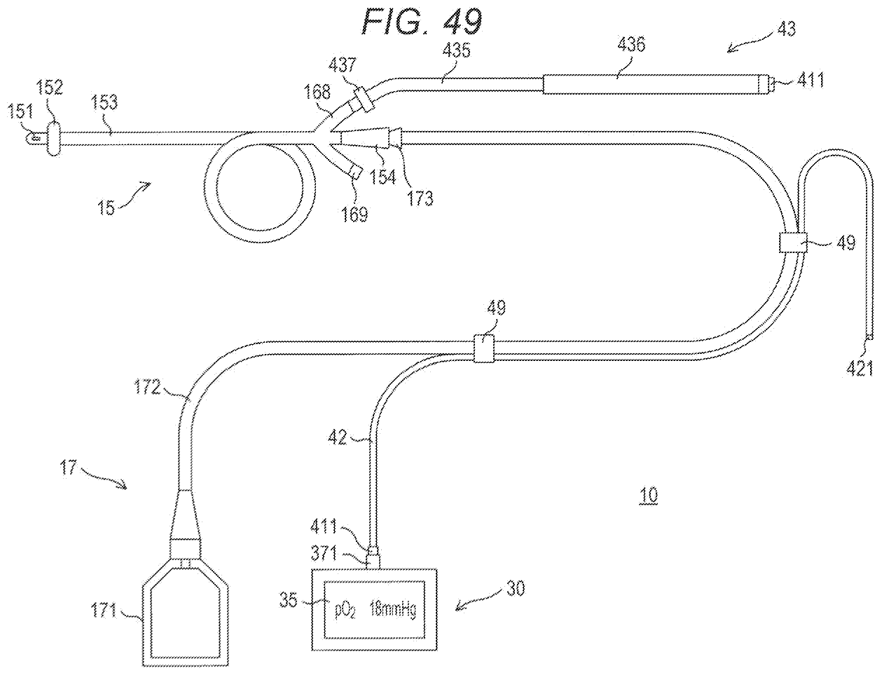

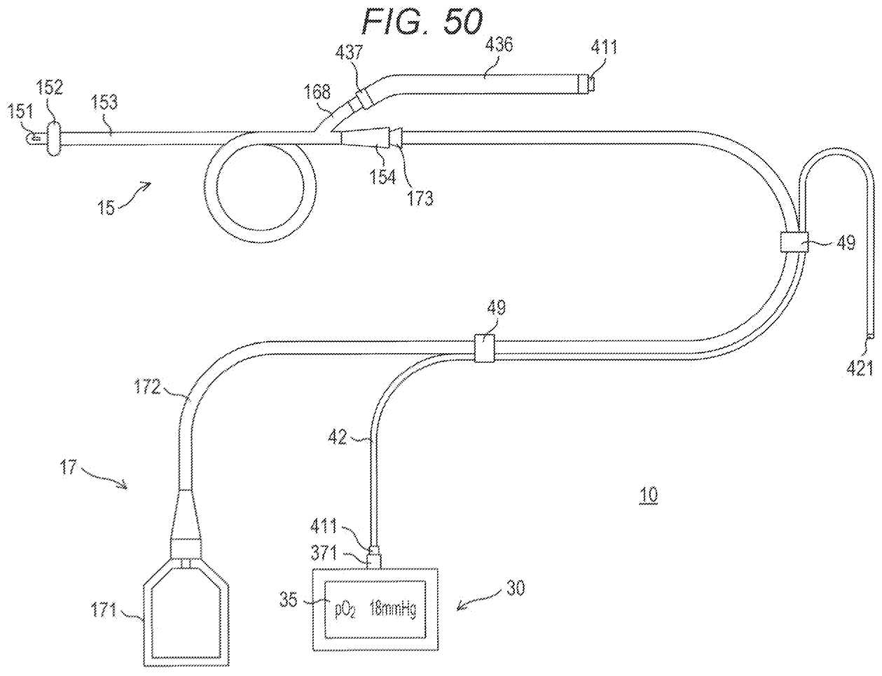

FIGS. 49 and 50 are explanatory views illustrating a method of using the measurement system 10 of Embodiment 8. An extension fiber 42 is connected to the measurement device 30. An optical fiber receptacle 421 is provided at one end of the extension fiber 42.

The user connects the measurement probe 14 and the urine collection bag 17. The user inserts the measurement probe 14 in the state described with reference to FIG. 48A into the probe port 168. The user fixes the mounting portion 437 to the probe port 168. Through the above process, the bladder indwelling catheter 15 and the measurement probe 14 are in a state illustrated in FIG. 49. At this time, the measurement probe 14 is inserted to the middle of the shaft 153.

The user inserts the shaft 153 into the urethra of the patient. Since the measurement probe 14 is not inserted into a distal end side of the shaft 153, the shaft 153 is in a state of being rather easily bent, and it is difficult to inflict pain on the patient. In a state where the distal end of the shaft 153 enters the inside of the bladder, the user inflates the balloon 152. When the balloon 152 is inflated, the shaft 153 does not come out of the urethra.

The user slides the inner tube 435 and the outer tube 436 to accommodate the inner tube 435 inside the outer tube 436 as illustrated in FIG. 50. By this operation, the measurement probe 14 is pushed into the shaft 153, and the light emitter 24 is disposed near the side hole 151. The adapter 43 desirably has the lock mechanism that fixes the inner tube 435 in the state of being accommodated in the outer tube 436. The adapter 43 has a similar degree of flexibility as the urine collection tube 172 even in a state where the inner tube 435 is accommodated in the outer tube 436 with the measurement probe 14 being inserted.

Thereafter, the user connects the optical fiber connector 411 and the optical fiber receptacle 421. As described above, a state illustrated in FIG. 47 is completed.

The resistance when the measurement probe 14 is inserted into the bladder indwelling catheter 15 increases as an inserted portion becomes longer. Thus, it is necessary for the user to push the measurement probe 14 with a stronger force as the final stage of insertion process approaches. According to the present embodiment, it is possible to help prevent buckling or the like at the final stage of the work of inserting the measurement probe 14 by using the adapter 43.

By using the extension fibers 42, it is possible to provide the measurement system 10 in which the measurement device 30 can be disposed at a position away from a bed of the patient. The user can select and use the extension fiber 42 having an appropriate length according to a layout of a hospital room or the like. Further, the extension fiber 42 may be connected to the optical fiber connector 411 in advance, or may be configured using the same optical fiber. Then, it is possible to prevent a mistake such as forgetting to connect, and the number of members can also be reduced.

### Embodiment 9

The present embodiment relates to the bladder indwelling catheter 15 to which the measurement probe 14 is fixed. Description of portions common to Embodiment 1 will be omitted.

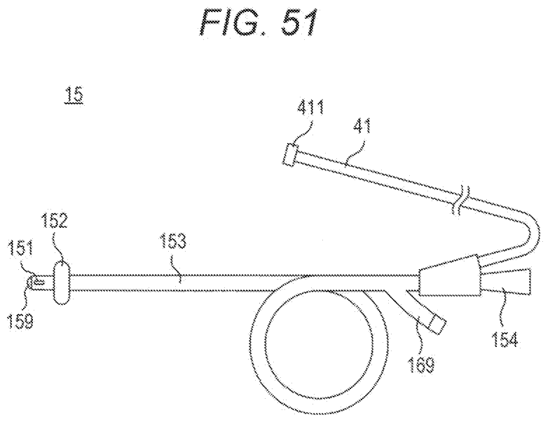

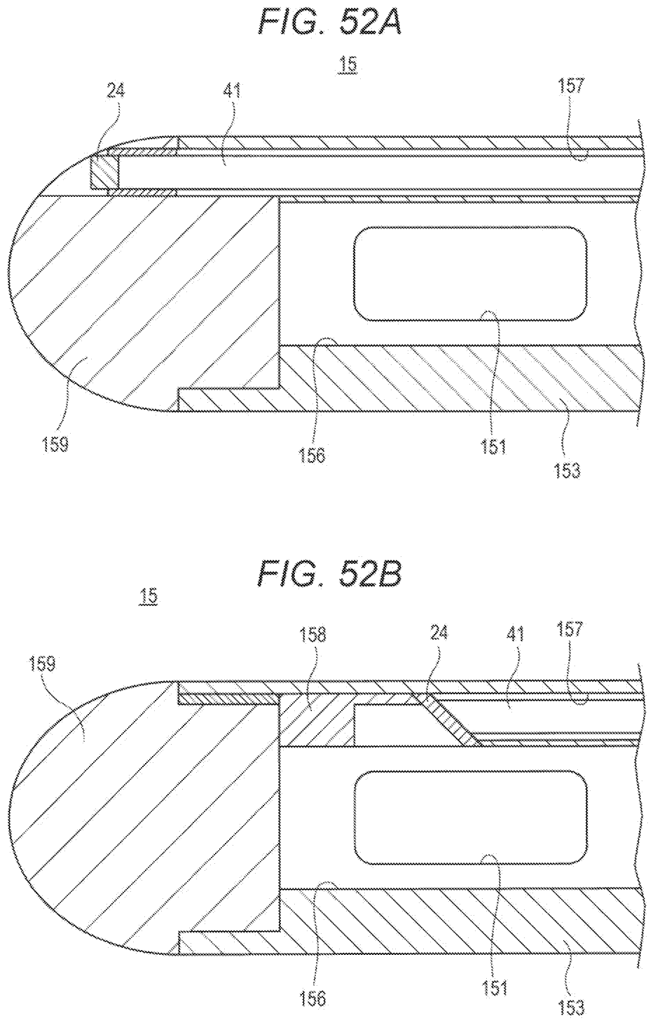

FIG. 51 is an explanatory view illustrating a configuration of the bladder indwelling catheter 15 of Embodiment 9. FIG. 52A is an enlarged cross-sectional view of a distal portion of the bladder indwelling catheter 15 of Embodiment 9.

In the present embodiment, a through-hole provided in the distal end member 159 communicates with the probe conduit 157, which is similar to Modification 7-4 described using FIG. 29. The probe conduit 157 and the urine passage 156 do not communicate with each other, which is different from Modification 7-4. The distal end member 159 and a distal portion of the measurement probe 14 are attached and fixed to each other. The adhesion fixing portion implements a function of a fixing portion of the present embodiment by fixing the measurement probe 14 in a state of being inserted into the probe conduit 157 or the like.

That is, the bladder indwelling catheter 15 of the present embodiment includes the optical fiber 41 to which the light emitter 24 is mounted and the optical fiber connector 411 in addition to the shaft 153, the urination funnel 154, and the balloon 152 as illustrated in FIG. 51.

According to the present embodiment, it is possible to provide the bladder indwelling catheter 15 that does not require work of inserting the measurement probe 14 into the bladder indwelling catheter 15 and enables preparation for use in a relatively short time.

### Modification 9-1

FIG. 52B is an enlarged cross-sectional view of a distal portion of the bladder indwelling catheter 15 of Modification 9-1. In the present modification, a support 158 is fixed to a distal end of the bladder indwelling catheter 15. The light emitter 24 having a plate shape is supported by the support 158. The light emitter 24 is disposed obliquely with respect to a central axis of the bladder indwelling catheter 15, and seals a distal portion of the probe conduit 157. An end surface of the optical fiber 41 is obliquely polished and is in contact with the light emitter 24.

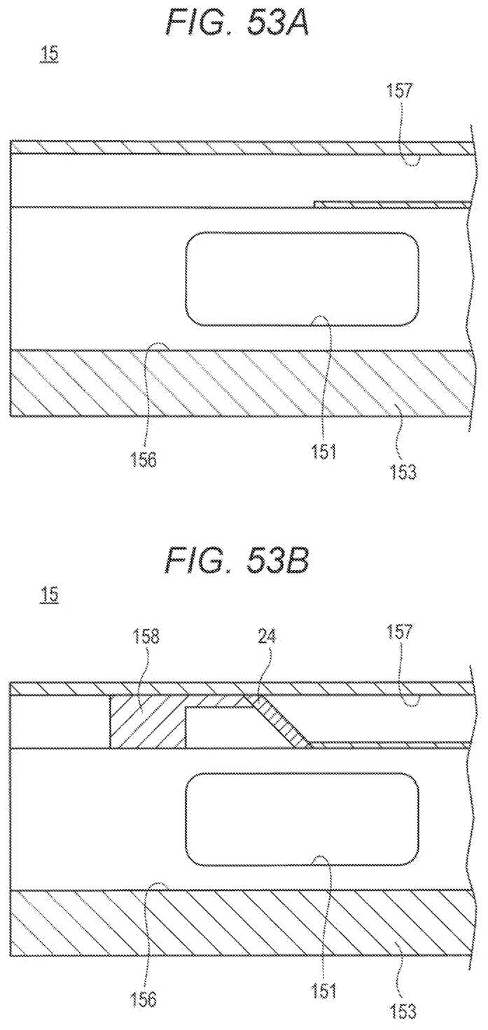

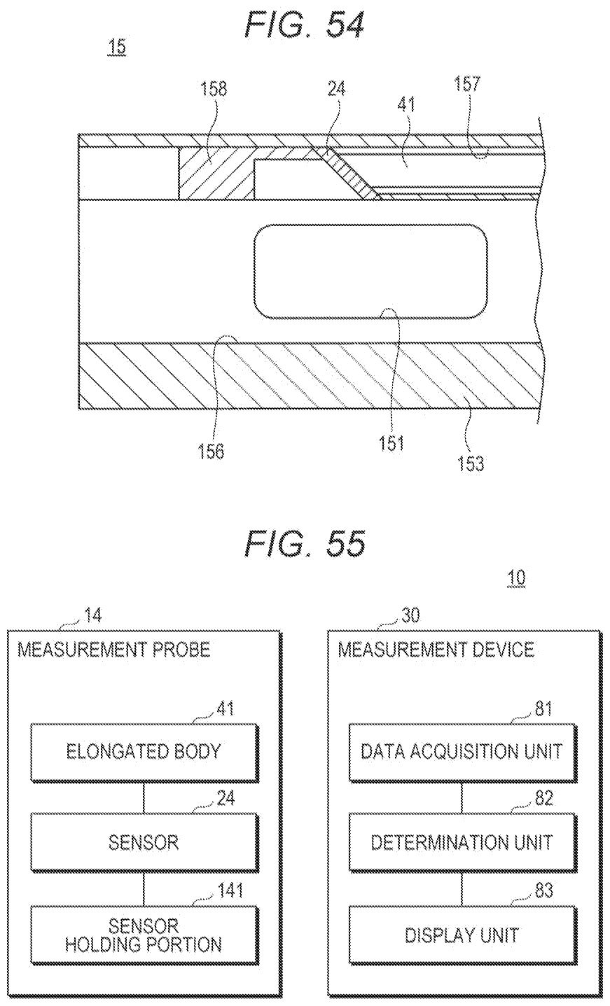

FIGS. 53A to 54 are explanatory views illustrating an assembly procedure of the bladder indwelling catheter 15 of Modification 9-1. Note that the process other than fixing the light emitter 24 and the optical fiber 41 is similar to that in an assembly procedure with the bladder indwelling catheter 15 in related art, and thus, the description of the process other than the fixing the light emitter 24 and the optical fiber 24 will be omitted.

The shaft 153 is a multi-lumen tube having three conduits of the urine passage 156, the probe conduit 157, and the balloon conduit 155. As illustrated in FIG. 53A, a wall between the urine passage 156 and the probe conduit 157 is removed at a distal portion of the shaft 153.

As illustrated in FIG. 53B, the distal portion of the probe conduit 157 is sealed by the light emitter 24 fixed to the support 158. As illustrated in FIG. 54, the optical fiber 41 whose distal end has been obliquely polished is inserted into the probe conduit 157. A translucent adhesive is applied to the distal end of the optical fiber 41, and the optical fiber 41 and the light emitter 24 are attached and fixed to each other.

Thereafter, the distal end member 159 is inserted into the distal portion of the bladder indwelling catheter 15 and fixed with an adhesive. As described above, the bladder indwelling catheter 15 of the present modification is completed.

### Embodiment 10

FIG. 55 is a functional block diagram of the measurement system 10 of Embodiment 10. The measurement system 10 includes the measurement probe 14 and the measurement device 30. The measurement probe 14 includes an elongated body 41 that can be inserted into a catheter 15 having a flow path 156, a sensor 24 that is fixed to the elongated body 41 and can detect a state of a fluid flowing in the flow path 156, and a sensor holding portion 141 that holds the sensor 24 at a predetermined position when the elongated body 41 is inserted into the catheter 15.

The measurement device 30 includes a data acquisition unit 81 that acquires data from the sensor 24 held by the sensor holding portion 141, a determination unit 82 that determines a condition of a patient in which the catheter 15 is indwelled on the basis of the acquired data, and a display unit 83 that displays the determined condition.

The technical features (configuration requirements) described in the embodiments can be combined with each other, and new technical features can be formed by the combination.

The detailed description above describes embodiments of a measurement probe, a catheter set, and a measurement system. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A measurement probe comprising:
an elongated body configured to be inserted into a catheter, the catheter including a flow path;
a sensor fixed to the elongated body and configured to detect a state of a fluid flowing in the flow path; and
a sensor holding portion configured to hold the sensor at a predetermined position when the elongated body is inserted into the catheter.

2. The measurement probe according to claim 1, wherein an output of the sensor is configured to change depending on an oxygen partial pressure in the fluid, a carbon dioxide partial pressure in the fluid, a hydrogen ion index of the fluid, an amount of potassium ions in the fluid, an amount of sodium ions in the fluid, an amount of chlorine ions in the fluid, a temperature of the fluid, or a flow rate of the fluid.

3. The measurement probe according to claim 1, wherein the sensor holding portion is configured to abut against an end of the flow path.

4. The measurement probe according to claim 1, wherein
the sensor includes a light emitter configured to emit light when the light emitter comes into contact with a component to be measured; and
the elongated body includes a light guide configured to guide radiation light emitted from the light emitter.

5. The measurement probe according to claim 4, wherein the radiation light is fluorescence.

6. The measurement probe according to claim 4, further comprising:
a light shield configured to prevent light excluding the radiation light from entering the light guide.

7. The measurement probe according to claim 4, wherein the light emission state of the light emitter is configured to change depending on each of a plurality of measurement items.

8. The measurement probe according to claim 1, further comprising:
a plurality of the sensors fixed to the elongated body; and
wherein the plurality of the sensors are fixed at different positions or a same position along a longitudinal direction of the elongated body.

9. The measurement probe according to claim 1, wherein the catheter is a bladder indwelling catheter having a urine passage, and the elongated body is configured to be inserted into the urine passage.

10. A catheter set comprising:
a catheter, the catheter including an opening provided on a distal end side, and a flow path that is provided between the opening and a rear end side and includes a large diameter portion disposed in the opening, a small diameter portion disposed on the rear end side, and a tapered portion disposed between the large diameter portion and the small diameter portion; and
a measurement probe, the measurement probe includes a sensor disposed on the tapered portion.

11. The catheter set according to claim 10, further comprising:
a check valve provided in the large diameter portion.

12. A measurement system comprising:
a measurement probe, the measurement probe includes:
an elongated body configured to be inserted into a catheter, the catheter having a flow path;
a sensor fixed to the elongated body and configured to detect a state of a fluid flowing in the flow path; and
a sensor holding portion configured to hold the sensor at a predetermined position when the elongated body is inserted into the catheter; and
a measurement device, the measurement device includes:
a data acquisition unit configured to acquire data from the sensor held by the sensor holding portion;
a determination unit configured to determine a condition of a patient in which the catheter is indwelled based on the acquired data; and
a display unit configured to display the determined condition.

13. The measurement system according to claim 12, wherein
the sensor includes a light emitter configured to emit light when the sensor comes into contact with a component to be measured;
the measurement probe includes a light guide configured to guide radiation light emitted from the light emitter;
the measurement device includes:
a light source configured to irradiate the light emitter with excitation light via the light guide; and
a light receiver configured to receive the radiation light guided by the light guide;
an optical analyzer configured to analyze the radiation light; and
the data acquisition unit is configured to acquire data related to a light emission state of the light emitter from the optical analyzer.

14. The measurement system according to claim 13, further comprising:
a light shield configured to prevent light excluding the radiation light from entering the light guide.

15. The measurement system according to claim 13, wherein the radiation light is fluorescence.

16. The measurement system according to claim 13, wherein
the sensor includes a plurality of the light emitters respectively corresponding to a plurality of measurement items; and
the light guide is configured to guide beams of the radiation light respectively emitted from the plurality of the light emitters in a mixed state.

17. The measurement system according to claim 13, further comprising:
a plurality of the sensors; and
wherein the light guide is configured to guide beams of the radiation light respectively emitted from the light emitters of the plurality of the sensors in a mixed state.

18. The measurement system according to claim 16, wherein the measurement device includes:
a spectroscopic unit configured to spectrally disperse the radiation light; and
a plurality of the optical analyzers configured to analyze beams of light dispersed by the spectroscopic unit.

19. The measurement system according to claim 16, wherein
the measurement device includes a filter configured to transmit a specific band of the radiation light; and
the optical analyzer is configured to analyze light transmitted through the filter.

20. The measurement system according to claim 12, wherein
the catheter has a non-communicating conduit that is disposed in parallel with the flow path and does not communicate with the flow path; and
the measurement system further comprising a non-wetted sensor inserted through the non-communicating conduit.
